(19) United States
(12) Patent Application Publication
(10) Pub. No.: US 2011/0263829 A1
(43) Pub. Date: Oct. 27, 2011

(54) CELL-PENETRATING, SEQUENCE-SPECIFIC AND NUCLEIC ACID-HYDROLYZING ANTIBODY, METHOD FOR PREPARING THE SAME AND PHARMACEUTICAL COMPOSITION COMPRISING THE SAME

(75) Inventors: Yong-Sung Kim, Suwon-si (KR); Myung-Hee Kwon, Suwon-si (KR); Woo Ram Lee, Siheung-si (KR)

(73) Assignee: AJOU UNIVERSITY INDUSTRY ACADEMIC COOPERATION FOUNDATION, Gyeonggi-do (KR)

(21) Appl. No.: 13/128,844

(22) PCT Filed: Nov. 11, 2009
(86) PCT No.: PCT/KR2009/006628

§ 371 Date: May 11, 2011

(30) Foreign Application Priority Data
Nov. 11, 2008 (KR) 10-2008-0111712

(51) Int. Cl. C07K 16/44; C07K 1/14

(52) U.S. Cl. 530/387.3; 530/387.5

(57) ABSTRACT

Disclosed are a cell-penetrating, base sequence-specific, nucleic acid-hydrolyzing antibody, a method of preparing the same, and a pharmaceutical composition comprising the same. The antibody can be prepared by modifying a particular site of a cell-penetrating, nucleic acid-hydrolyzing antibody which lacks substrate specificity to impart sequence specificity thereto without alteration in nucleic acid-hydrolyzing ability. The antibody, when penetrating into cells by itself or ectopically expressed within cells, binds specifically to single- or double-stranded nucleic acid targets and hydrolyzes them, thus downregulating the expression of the targeted genes.

### TECHNICAL FIELD

The present invention relates to a nucleic acid-hydrolyzing antibody with cell-penetrating ability and base sequence specificity, as the next-generation gene silencing technique overcoming the problems that conventional siRNA technique has. More particularly, the present invention relates to a nucleic acid-hydrolyzing antibody, prepared by modifying a particular site of a cell-penetrating, nucleic acid-hydrolyzing antibody which lacks substrate specificity to impart sequence specificity thereto without alteration in nucleic acid-hydrolyzing ability, which when penetrating into cells by themselves or ectopically expressed within cells, can bind specifically to single-stranded/double-stranded nucleic acid targets and hydrolyze them, thus down-regulating the expression of the targeted genes. Also, the present invention is concerned with a method of preparing the antibody and a pharmaceutical composition comprising the antibody.

### BACKGROUND ART

There are three major classes of the biopolymer that play important roles in the central dogma of molecular biology: DNA, RNA and protein. The transcription of DNA into RNA needs the help of certain proteins and ribosomes. These proteins are associated with DNA at specific sites to start transcription. The resulting RNA finds its way to a ribosome where it is translated into proteins. A typical method for examining what functions the protein products do comprises the removal of the proteins from the biosystem. A difference between behaviors of a living organism with and without the protein of interest accounts for the role which it plays in the biosystem. However, it is difficult to control the expression level of a protein of interest at discretion in living organisms. Recently, various nucleic acid-based approaches to the control of protein expression which specifically recognize and hydrolyze particular regions of targeted RNA (mRNA included) have been developed, including antisense oligonucleotides, interference RNA (RNAi), ribozyme, DNAzymes, etc. (Scherer et al., Nature Biotechnology, 21:1457-1465, 2003; Tafech et al., Current Medical Chemistry, 13:863-881, 2006). Particularly, RNAi, found in 1998, is now readily available and makes the knockdown of RNA more convenient than dose the prior art (Fire A et al., Nature, 391:806-811, 1998; Scherer et al., Nature Biotechnology, 21:1457-1465, 2003). So-called siRNA (small interfering RNAs), double-stranded (ds) RNAs 21-23 bp in length, is central to RNAi. These small RNAs with certain sequences, whether generated inside or transferred from the outside, can bind to and hydrolyze specific mRNAs to downregulate the expression of targeted proteins within cells (called gene knockdown). Although its principle has been established for not yet 10 years, the siRNA technique is now the most widely applied for decreasing the expression level of proteins in plant/animal cells. However, there are several problems with RNAi upon practical application. One representative example is an off-target effect which is generated when the RNA, even 21-mer in length, cannot pair with the target. Further, siRNA-induced gene knockdown is significantly decreased or is not elicited if siRNA differs from the target in even one or two base pairs. In addition, RNAi may be effective operated in a specific region of a target gene, but does not work in the other range at all in many cases. Besides, including undesired immune response, improper cellular delivery, nuclease susceptibility, etc. act as inhibitive factors in the practical application of siRNAs (Scherer L J et al., Nat Biotechnol, 21:1457-1465, 2003; Tafech A et al., Curr Med Chem, 13:863-881, 2006).

Since the first finding in the serum of a patient with systemic lupus erythematosus (SLE) in 1957, nucleic acid (DNA/RNA)-binding antibodies, a kind of autoantibodies, are detected in autoimmune disease patients or mice (Robbins W et al., Proc. Soc. Exp. Biol. Med., 96(3): 575-9, 1957). Many anti-nucleic acid autoantibodies are practically found in patients with SLE or multiple sclerosis. Generally, they bind to nucleic acids with the lack of sequence specificity (Jang Y J et al., Cell. Mol. Life Sci., 60(2):309-20, 2003; Marion T N et al., Methods 11:3-11, 1997). It is reported that sera from SLE patients and the SLE mouse model MRL^{-lpr/lpr} have high titers of anti-nucleic acid antibodies and studies on autoantibodies have been conducted mainly in patients with autoimmune diseases (Dubrivskaya V et al., Biochemistry (Mosc), 68(10):1081-8, 2003).

In 1992, a nucleic acid-binding antibody with ability to hydrolyze nucleic acids was first found (Shuster A et al., Science, 256 (5057):665-7, 1992). Since then, biochemical studies have been focused thereon (Nevinsky G et al., J. Immunol. Methods, 269(1-2):235-49, 2002). Studies on nucleic acid-hydrolyzing antibodies have been thus advanced in terms of biochemistry, but have remained in the initial phase in terms of the antibody engineering aspect, such as improvements in stability, affinity and specificity for various applications of antibodies (Cerutti M et al., J. Biol. Chem., 276(16): 12769-73, 2001; Kim Y R et al., J. Biol. Chem., 281(22): 15287-95, 2006).

Binding between antibodies and nucleic acids and between non-antibody proteins and nucleic acids is disclosed in several reports. First, a zinc finger, a non-antibody protein, is representative of naturally occurring DNA binding motifs, like leucine zipper and helix-turn-helix (Jamieson A et al., Nat. Rev. Drug Discov., 2(5):361-8, 2003). A zinc finer, a small protein domain composed of about 20-30 amino acid residues, coordinates a zinc ion (Zn²⁺) with a usual combination of two cysteines and two histidine residues from four different directions. Being practically responsible for DNA binding, the alpha-helix of the zinc finer is associated with the major groove of DNA while interacting with three bases. The interacting triplet of DNA differs depending on the amino acid sequence of the zinc finger. Accordingly, when modified in the alpha-helix without a conformational change, a zinc finer can recognize a new base sequence which is different from the prior one. Since 1999 in which specific fingers were successfully modified for 16 GNN triplets (Segal D et al., Proc. Natl. Acad. Sci. USA, 96(6):2758-63, 1999), extensive research have been performed to establish a method for modifying substrate specificity (Caroli D et al., Nat. Protoc. 1(3):1329-41, 2006). Because they have only an ability to bind to nucleic acids, however, the modified zinc fingers require an additional modification for association with a nucleic acid-hydrolyzing enzyme (Mani M. et al., Biochem. Biophys. Res. Commun., 334(4):1191-7, 2005).

A second approach is an empirical method which takes advantage of the DNA-binding domain of human papillomavirus (HPV) E2 protein (E2C) in binding a target DNA (M. Laura et al., J. Bio. Chem., 276(16): 12769-73, 2001). A DNA-E2C complex is injected into a mouse to produce anti-DNA antibodies through somatic hypermutation. In this regard, the mouse should recognize the DNA as an antigen. For this, first, a DNA-protein (E2C) complex is intra-abdominally injected into a mouse to induce an immune response. When the DNA-E2C complex is repetitively injected for a certain time to amplify the immune response, antibodies with specificity for the DNA of the injected DNA-E2C complex are produced through somatic hypermutation. After the amplification, the resulting antibodies are isolated from the mouse. From among the isolates capable of specifically binding to the DNA, an antibody showing highest affinity for the DNA can be selected by reacting them with the DNA of interest.

A rational design provides a third way to describe the binding of antibodies to nucleic acids. In this method, a β-sheet of human γ-B-crystallin is used to generate a universal binding site through randomization of solvent-exposed amino acid residues selected according to structural and sequence analyses (Hilmar E. et al., J. Mol. Biol., 372:172-85, 2007). As a general rule, an antibody is structurally divided into frameworks and flexible, sequence-variable CDRs (complementarity-determining regions). The flexibility of CDRs allows the antibody to form an induced-fit with an antigen. An alternative mechanism for high specificity and affinity is a lock and key model. In this regard, because the protein already forms a complementary structure to retain a high affinity for the substrate, it can maintain essential antibody stability and undergoes no conformational changes upon binding and thus can more strongly bind with the substrate (Jackson R. et al., Protein Sci., 8:603-13, 1999).

Recent trends in protein engineering and library selection are therefore shifted from the CRDs to the framework. In fact, first, a functional Zn-binding site is introduced on the surface of the β-barrel of mammal serum retinol-binding protein using a rational design (Muller H. et al., Biochemistry, 33: 14126-35, 1994). Next, binding activity is imparted to the β-sheet of a cellulose-binding domain derived from the CBH (cellobiohydrolase) Cel7A of Trichoderma reesei by mutation (Lehtio J. et al., Proteins: Struct. Funct. Genet., 41:316-22, 2000). Another study is concerned with an ankyrin repeat protein composed of two antiparallel α-helices and one β-turn (Binz H. et al., J. Mol. Biol., 332:489-503, 2003).

Gene silencing by targeting specific genes for degradation at the mRNA level so as to downregulate the expression of the proteins encoded thereby is known to be an invaluable tool for gene function analysis as well as a powerful therapeutic strategy for human diseases, including cancer and viral infections. Conventional gene silencing techniques are, for the most part, based on the ability of nucleic acids complementary to single-stranded nucleic acids to inhibit the translation of mRNA (Scherer L J et al., Nat Biotechnol, 21:1457-65, 2003; Tafech A et al., Curr Med Chem, 13:863-81, 2006). Of them is representative siRNA (small interfering RNA). However, siRNA suffers from the disadvantages of lacking cell-penetrating ability, being low in stability due to RNase susceptibility, being likely to acting on off-targets, and inducing immunogenicity.

As described above, the conventional gene silencing technique such as that using siRNA can cause a specific gene to decrease in expression level, but requires an additional modification for ability to hydrolyze nucleic acids in such a way that it is conjugated with a nuclease hydrolyzing enzyme.

Currently marketed drugs and drug development under current study are based on small molecules, proteins and monoclonal antibodies. Most of them are designed to bind to proteins the activity of which is in turn controlled to elicit pharmaceutical effects. Particularly, almost all monoclonal antibodies and proteins target membrane proteins or extracellular proteins. In spite of a great number of different genes associated with various diseases, drug development has been focused on protein targets so far, resulting in a very limited number of drugs. If developed, drugs which can control diseases at an RNA or DNA level, but not at a protein level, that is, which can target intracellular RNA or DNAs may cover a wider range of diseases. Further, nuclease-hydrolyzing antibodies which can penetrate into cells and recognize particular base sequences may be highly likely to be developed into next-generation gene-silencing and anti-viral agents.

Therefore, there is a need for an antibody that can itself penetrate into cells without external protein delivery systems, and can specifically bind to and hydrolyze single-stranded/double-stranded target nucleic acids of particular sequences.

### DISCLOSURE OF INVENTION

### Technical Problem

Leading to the present invention, intensive and thorough research into gene silencing, conducted by the present invention, with the aim of overcoming the problems encountered in the prior art, resulted in the finding that a cell-penetrating, nucleic acid-hydrolyzing antibody which lacks substrate specificity can be imparted with specificity for single- or double-stranded targets without alteration in nucleic acid-hydrolyzing ability by modifying a particular site thereof and that the modified antibody, when penetrating into cells by themselves or expressed within cells, can bind specifically to single- or double-stranded nucleic acid targets and hydrolyze them, thus down-regulating the expression of certain genes.

### Solution to Problem

It is therefore an object of the present invention to provide a nucleic acid-hydrolyzing antibody which can penetrate into cells, bind specifically to a single-stranded/or double-stranded nucleic acid target of a particular base sequence, and hydrolyze it.

It is another object of the present invention to provide to a method of preparing the nucleic acid-hydrolyzing antibody.

It is a further object of the present invention to provide a pharmaceutical composition comprising the nucleic acid-hydrolyzing antibody.

### BRIEF DESCRIPTION OF DRAWINGS

FIG. 1 schematically shows the nucleic acid-hydrolyzing antibody of the present invention with regard to formats thereof (A) and the hydrolyzation of single- or double-stranded target nucleic acids of particular base sequences (B).

FIG. 2 is a schematic illustration of a procedure in which after being translocated into the cytoplasm by cellular penetration or cytosolically expressed by transfection, the nucleic acid-hydrolyzing antibody with sequence specificity of the present invention acts to specifically recognize and hydrolyze an exogenous target gene carried by external matter (e.g., virus) or an endogenous target mRNA, thereby inhibiting viral proliferation or protein expression.

FIG. 3 is a view showing the tertiary structure of 3D8 VL (A) and the amino acid sequences and base sequences of the c- (residues 41-45), c′- (residues 50-54) and fβ-strands (residue 90-94) constituting the putative DNA/RNA recognition site of 3D8 VL WT, and the NNB codons used for mutation (B).

FIG. 4 shows the construction of a library of nucleic acid-hydrolyzing antibody on the template of 3D8 VL 4M (A), the expression of the library on yeast cell surfaces following cotransformation with a yeast display vector (pCTCON) by electroporation (B) and FACS analysis of the expression levels of the library (C).

FIG. 5 shows the representative screening procedures for the isolation of 3D8 VL variants preferentially binding to the two ss-DNA target substrates, G₁₈ (A) and Her2₁₈ (B), from the yeast surface-displayed 3D8 VL library.

FIG. 6 is a view showing the amino acid sequence alignment of 3D8 VL WT and 3D8 VL 4M variants selected against the target 18-bp ss-DNAs, G₁₈ (4MG1-4MG6) and Her2₁₈ (4MH1-4MH5), focusing on the 15 randomized positions on the c- (residues 41-45), c′- (residues 50-54) and f-β-strands (residues 90-94).

FIG. 7 shows data for SDS-PAGE analysis of the purified 11 variants (A), and size-exclusion HPLC (B) and Far-UV CD (circular dichroism) spectroscopy (C) of the representative variants (4MG3, 4MG5, 4MH2), compared with 3D8 VL WT and 4M.

FIG. 8 shows results of the agarose gel electrophoresis for DNA-hydrolyzing activity of the 11 variants (A) and for RNA-hydrolyzing activity of 4MG3, 4MG5 and 4MH2 (B).

FIG. 9 shows plots of the enzyme kinetics of the 3D8 VL WT and 3D8 VL 4M and the variants (4MG3, 4MG5, 4MH2) as functions of the concentrations of FRET substrates (A₁₈, T₁₈, C₁₈, (G₄T)₃G₃, Her2₁₈, N₁₈) from 16 nM to 2 μM.

FIG. 10 is of schematic diagrams showing plasmids for the cytosolic expression of 3D8 VL wild-type and the variants (4MG3, 4MG5) (A, pcDNA3.1), GFP (B, pEGFP-N1), GFP (C, pG₁₈-EGFP in which G₁₈ is located in the N-terminal upstream of EGFP), and EGFP (D, pHer2₁₈-EGFP in which Her2₁₈ is located in the N-terminal upstream of EGFP).

FIG. 11 shows target gene silencing activity of selected 3D8 VL variants, which were ectopically co-expressed with target-sequence carrying EGFP in HeLa cells. HeLa cells were untransfected or transfected with EGFP encoding plasmids (intact EGFP, G₁₈-EGFP, or Her2₁₈-EGFP) alone or together with plasmids encoding 3D8 VLs (WT, G₁₈-selective 4MG3 and 4MG5, and Her2₁₈-selective 4MH2), as indicated in the panels, and then monitored for EGFP expression by flow cytometry (A), confocal fluorescence microscopy (B), Western blotting (C, D) and RT-PCR (E, F).

FIG. 12 shows the effect of Her2₁₈ base sequence-specific, nucleic acid-hydrolyzing 4MH2 in HeLa cells on Her2 gene expression, which was analyzed for its mRNA level by RT-PCR (A) and for its protein expression level by Western-blotting (B).

FIG. 13 shows data demonstrating that 3D8 VL variants penetrate into living cells and localize dominantly in the cytosol. (A) FACS data on the cellular internalization of 3D8 VL wild-type and the variants (4MG3, 4MG5, 4MH2) into human cervical carcinoma cells (HeLa) and human breast carcinoma cells (SK-BR3). (B) Confocal fluorescence microscopy of internalization and subcellular localization of 3D8 VLs in HeLa cells. (C) FACS data analyzed for effect of pre-treatment of soluble heparin or specific endocytosis inhibitors on the cellular uptakes of 3D8 VL wild-type and the variants (4MG3, 4MG5, 4MH2).

FIG. 14 shows target gene silencing activity of cell-penetrating 3D8 VL variants in HeLa cells expressing exogenous targeted genes. HeLa cells were untransfected or transfected with plasmids encoding EGFP or G₁₈-EGFP, either untreated or treated with 3D8 VL WT and G₁₈-selective 4MG3 and 4MG5, and analyzed by flow cytometry (A), RT-PCR (B), and Western blotting (C). Her2-negative HeLa cells were untransfected or transfected with a plasmid encoding the full-length Her2 gene, and were either untreated or treated with 3D8 VL WT and Her2₁₈-selective 4MH2. Her2 expression was analyzed by RT-PCR (D) and Western blotting (E).

FIG. 15 shows the viability of the Her2-overexpressing human breast carcinoma cells (SK-BR-3, MDA-MB-231) or Her2-negative human cervical carcinoma cells (HeLa) treated with the Her2₁₈-specific 4MH2 variant, analyzed by MTT assay (A) and FACS (B).

FIG. 16 shows cell-penetrating Her2₁₈-selective 4MH2 knocks-down endogenous Her2 expression in Her2-overexpressing SK-BR-3 cells. Her2 expression was monitored at the cell-surface by flow cytometry (A), at the mRNA level by RT-PCR (B), and at the protein level by Western blotting (C).

### BEST MODE FOR CARRYING OUT THE INVENTION

In accordance with an aspect thereof, the present invention pertains to a nucleic acid-hydrolyzing antibody which possesses the cell-penetrating ability and can bind specifically to and hydrolyze single- or double stranded target nucleic acids of particular base sequences.

In accordance with another aspect thereof, the present invention pertains to a method of preparing a cell-penetrating, sequence-specific, and nucleic acid-hydrolyzing antibody, comprising:

1) constructing a library of genes on a template of a cell-penetrating nucleic acid-hydrolyzing antibody which lacks substrate specificity;

2) expressing the library gene constructed in step 1) on a cell surface by use of a surface-displaying vector to produce a library of proteins; and

3) selecting from the library of proteins expressed in step 2) a variant which binds specifically to a nucleic acid target of a particular base sequence.

In accordance with a further aspect thereof, the present invention pertains to a pharmaceutical composition comprising the nucleic acid-hydrolyzing antibody.

Hereinafter, a detailed description will be given of the present invention.

Constructed as a result of antibody engineering by modifying a particular region of a nucleic acid-hydrolyzing antibody which possesses the cell-penetrating ability but not of substrate specificity, the nucleic acid-hydrolyzing antibody according to the present invention is further imparted with sequence specificity. When it penetrates into the cytoplasm or is expressed within cells, the nucleic acid-hydrolyzing antibody of the present invention can bind specifically to and hydrolyze a single- or double-stranded nucleic acid target of a particular base sequence to downregulate the expression of the particular gene.

The engineered, nucleic acid-hydrolyzing antibody of the present invention has amino acid sequences of SEQ ID NOS: 14 to 24 with preference for SEQ ID NOS: 16, 18 and 21. The base sequences of nucleic acid-hydrolyzing antibody of the present invention are represented by SEQ ID NOS: 25 to 35, with preference for SEQ ID NOS: 27, 29 and 32.

The nucleic acid-hydrolyzing antibody of the present invention may be in its entirety or may be a functional fragment. The antibody in its entirety may be in the form of a monomer or a multimer in which two or more entire antibodies are associated with each other and include the entire IgG. As used herein, the term “a functional fragment” with respect to an antibody is intended to refer to an antibody fragment having a heavy chain variable region and a light chain variable region which can recognize the substantially same epitope as does the entire antibody. Examples of the functional fragment of the antibody include single domain of the heavy chain variable region, single domain of the light chain variable region, single-chain variable fragments (scFv), (scFv)₂, Fab, Fab′, F(ab′)₂, diabody, and disulfide-stabilized variable fragments (dsFv), but are not limited thereto, with single domain of the light chain variable region being preferred.

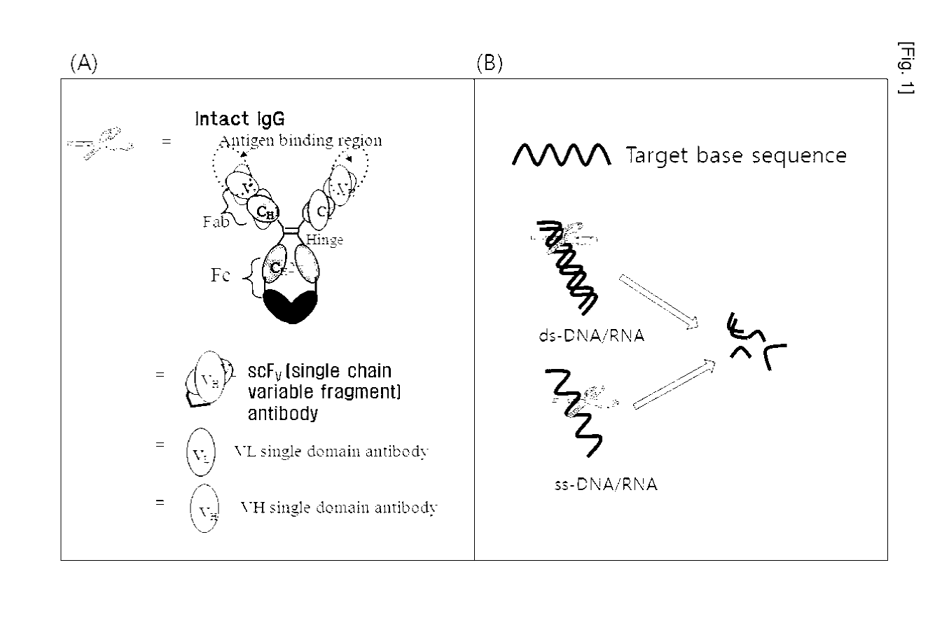

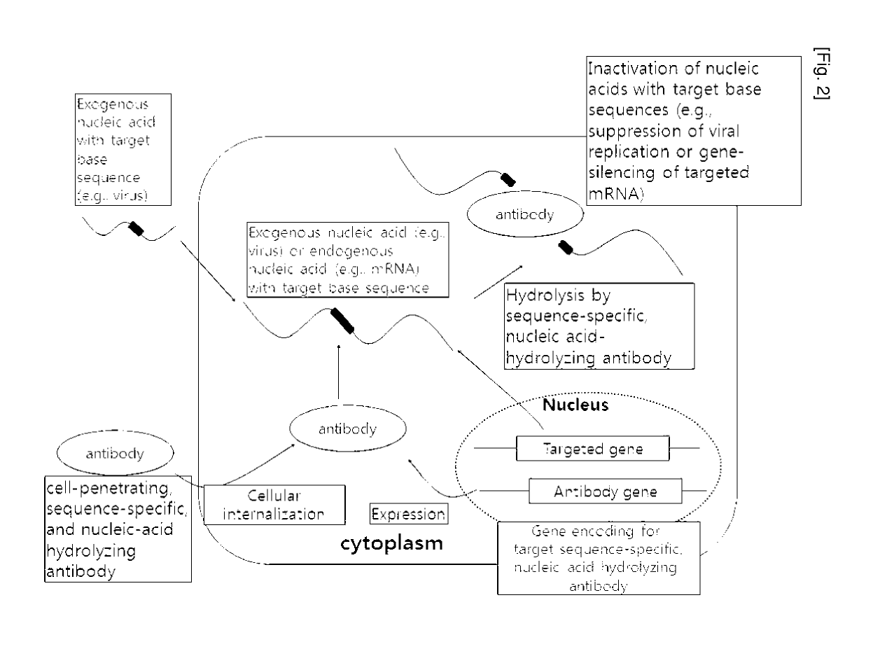

With reference to FIG. 1, the nucleic acid-hydrolyzing antibody of the present invention is schematically illustrated with regard to formats thereof (A) and the hydrolyzation of single- or double-stranded target nucleic acids of particular base sequences (B). With reference to FIG. 2, a schematic illustration is given of a procedure in which after being translocated into the cytoplasm by cellular penetration or cytosolically expressed by transfection, the nucleic acid-hydrolyzing antibody with sequence specificity of the present invention acts to specifically recognize and hydrolyze an exogenous target gene carried by external matter (e.g., virus) or an endogenous target mRNA, thereby inhibiting viral proliferation or protein expression.

Next, turning to the method of preparing the nucleic acid-hydrolyzing antibody of the present invention, its description is given in a stepwise manner as follows.

Step 1) is to synthesize a library of genes using a cell-penetrating, nucleic acid-hydrolyzing antibody lacking sequence specificity as a template. As the antibody which can penetrate into cells and hydrolyze nucleic acids, but lacks sequence specificity, 3D8 VL 4M or its variant is preferred. A structural analysis of 3D8 VL allowed a putative nucleic acid-binding site composed of the c-, the c′- and the fβ-strand. This putative binding site is randomized with the degenerated NNB codons (N=A/T/C/G, B=C/G/T) to construct on yeast cell surfaces library with mutations at all residues.

Step 2) is of the construction of the library on a cell surface. The amplified 3D8 VL library gene are co-transformed together with a display vector into cells by electroporation to construct library of 3D8 VL on yeast cell surfaces. Examples of the display vector useful in the present invention include phage display, bacterial display, ribosome display, RNA display and yeast cell display vectors, but are not limited thereto. In the present invention, a yeast display vector is employed for library construction. The library was expressed well on yeast cell surfaces.

In Step 3), the 3D8 VL 4M antibody library is screened against target nucleic acid sequences to select 3D8 VL variants specifically binding thereto. In this regard, 5′-biotinylated target nucleic acids are used to analyze the antibody library for specific affinity therefor. The target nucleic acids may be endogenous or exogenous. Preferably, endogenous nucleic acids may be nucleic acids coding for proteins which are overexpressed in specific response to cancer cells. A preferred exogenous nucleic acid is a viral genomic nucleic acid or a nucleic acid coding a viral protein. In greater detail, the antibody libraries are screened against two 5′-biotinylated DNA targets (G₁₈, Her2₁₈) during which they are analyzed for affinity for the respective targets (G₁₈, Her2₁₈) in comparison with off-target nucleic acids using MACS and FACS. Based on the analysis, variants with strong specificity for the targets (G₁₈, Her2₁₈) are selected. As a result, six variants, 4MG1, 4MG2, 4MG3, 4MG4, 4MG5 and 4MG6, were selected for the single-stranded DNA target (G₁₈) while five variants 4MH1, 4MH2, 4MH3, 4MH4 and 4MH5 were observed to have strong affinity for the single-stranded DNA target (Her2₁₈). These 11 variants in total have amino acid sequences of SEQ ID NOS: 14 to 24, respectively, with SEQ ID NOS: 16(4MG3), 18(4MG5) and 21(4MH2) being preferred. Correspondingly, the base sequences of the 11 variants are represented by SEQ ID NOS: 25 to 35, respectively. Accordingly, SEQ ID NOS: 27(4MG3), 29(4MG5) and 32(4MH2) are preferred. The selected variants are purified with greater than 90% purity and exist as soluble monomers with the secondary structures retained therein. Also, the 3D8 VL variants exhibited 10-100-fold greater K_{D} for their respective target substrates 4MH2 for Her2₁₈ and 4MG3 and 4MG5 for G₁₈ than off-targets because the affinity thereof greatly increases for the target substrates, but remains unchanged for off-targets. In addition, showing higher Vmax values for substrate degradation rate compared to 3D8 VL WT and 3D8 VL 4M, the variant antibodies recognize, specifically bind to and hydrolyze target nucleic acid sequences faster. Therefore, the variants according to the present invention are adapted to have sequence specificity with the retention of the ability to hydrolyze DNA and RNA.

The expression level of EGFP (enhanced green fluorescent protein) without the target sequence of G₁₈ or Her2₁₈ was affected neither by variants of the present invention nor by 3D8 VL wild-type. Meanwhile, cells cotransfected with vectors carrying the 3D8 VL variant of the present invention together with a vector carrying EGFP with G₁₈ or Her2₁₈ target sequence at the N-terminus expressed much lower EGFP signals than cotransfected with the 3D8 VL wild-type. It strongly suggested that the variants expressed within the cells hydrolyzed the mRNAs carrying the target sequences such as G₁₈-EGFP mRNA and Her2₁₈-EGFP mRNA, thus decreased the expression level of GFP. When expressed within cells, nucleic acid-hydrolyzing antibodies which are mutated to recognize target base sequences can hydrolyze mRNA containing target base sequences and thus downregulate the expression of the protein encoded by the mRNA. The variants of the present invention are demonstrated to have sequence specific, nucleic acid-hydrolyzing ability when they were ectopically expressed in the cells.

In addition, the variants of the present invention are found to penetrate into human cervical carcinoma cells (HeLa) and human breast carcinoma cells (SK-BR3) to an extent similar to that of the 3D8 VL wild-type. The internalization of the variants into cells proceeds to similar extents between cells pretreated with chlorpromazine for inhibiting clathrin-dependent endocytosis and with cytochalasin D for inhibiting macropinocytosis. In contrast, the cell-penetrating ability of the variants is remarkably decreased upon the pretreatment of cells with heparin for interfering with the electrical interaction of the positively charged variants with the negatively charged cell surface proteoglycan (heparansulfate) or upon pretreatment with methyl-β-cyclodextrin (MβCD) for inhibiting caveolae/lipid raft endocytosis, demonstrating that the variants are introduced into the cells through the caveolae/lipid raft endocytic pathway following electrical interaction with abundant proteoglycans on cell surfaces.

Further, the variants according to the present invention show low cytotoxicity against human breast carcinoma cells (SK-BR-3, MDA-MB-231) or human cervical carcinoma cells (HeLa). Particularly, the viability of Her2-overexpressing SK-BR-3 or MDA-MB-231 cells is significantly decreased by the nucleic acid-hydrolyzing 4MH2 with Her2 sequence specificity because of its strong cytotoxicity. This result is attributed to the fact that 4MH2 downregulates Her2 expression, which is coincident with the previous report that Her2-overexpressing cells decreases in viability with the decreasing of Her2 expression. At this time, the cell death was observed to show an apoptotic pattern (Annexin V positive).

As described above, the nucleic acid-hydrolyzing antibodies in accordance with the present invention are prepared by modifying a particular site of a cell-penetrating, nucleic acid-hydrolyzing antibody which lacks substrate specificity to impart sequence specificity thereto without alteration in nucleic acid-hydrolyzing ability. The engineered nucleic acid-hydrolyzing antibodies, when penetrating into cells by themselves or expressed within cells, bind specifically to single-stranded/double-stranded nucleic acid targets and hydrolyze them, thus down-regulating the expression of certain genes. Therefore, the nucleic acid-hydrolyzing antibodies according to the present invention can be an alternative to or a substitute for conventional gene silencing techniques such as siRNA. Particularly, the nucleic acid-hydrolyzing antibodies of the present invention can downregulate the expression of target proteins or the proliferation of target genomes at RNA or DNA levels, but not at protein levels, by binding specifically to and hydrolyzing RNA or DNA, so that they are useful as therapeutics for cancers and viral diseases. Accordingly, the nucleic acid-hydrolyzing antibodies of the present invention may be developed into novel anticancer drugs or anti-viral drugs with the anticipation of making inroads into the market.

In accordance with a further aspect thereof, the present invention pertains to a pharmaceutical composition comprising as an active ingredient the nucleic acid-hydrolyzing antibody of the present invention alone or in combination with at least one conventional anti-cancer or anti-viral ingredient.

For use in practical administration, the pharmaceutical composition may comprise at least one pharmaceutically acceptable vehicle in addition to the active ingredient. Examples of the pharmaceutically acceptable vehicle include biological saline, sterile water, Ringer's solution, buffered saline, dextrose solutions, maltodextrin solution, glycerol, ethanol, etc. Optionally, other typical additives such as antioxidants, a buffer, bacteriostatic agents, etc. may be added to the pharmaceutical composition of the present invention. The composition may be formulated into injections such as aqueous solutions, suspensions, emulsions, etc., pills, capsules, granules or tablets using diluents, dispersants, surfactants, binders, and/or lubricants. In addition, the composition may be formulated into suitable dosage forms according to a method well known in the art or the method disclosed in Remington's Pharmaceutical Science (latest), Mack Publishing Company, Easton Pa.

The composition of the present invention may be orally or non-orally (intravenously, subcutaneously, intra-abdominally, or locally) administrated. Its dose varies depending on the weight, age, gender, health condition, and diet of patient, time of administration, administration route, excretion rate, severity of diseases, etc. The nucleic acid-hydrolyzing antibody is administrated at a daily dose of from about 0.01 to 10 mg/kg and preferably at a daily dose of from 1 to 5 mg/kg once or in multiple doses a day.

In order to suppress the expression of pathogenic proteins or the proliferation of viral genes, the composition of the present invention may be used alone or in combination with surgery, hormonal therapy, chemical therapy or biological response regulators.

### MODE FOR THE INVENTION

A better understanding of the present invention may be obtained through the following examples which are set forth to illustrate, but are not to be construed as the limit of the present invention.

### Example 1

### Design of 3D8 VL 4M Antibodies

1. Expression of 3D8 VL 4M Antibody on Yeast Cell Surface

The first step of engineering a 3D8 VL antibody into a sequence-specific, nucleic acid-hydrolyzing one was to display the antibody on yeast cell surfaces. The antibody was the 3D8 VL 4M which was higher in DNA/RNA hydrolyzing activity than was the wild-type (WT). 3D8 VL 4M had four mutations of Q52R, Y55H, W56R, and H100A. In order to express the 3D8 VL 4M antibody on yeast cell surfaces, a 3D8 VL 4M gene was subcloned from the E. coli expression vector pET23M 3D8 VL 4M into the yeast cell surface display vector pCTCON. For the amplification of the 3D8 VL 4M gene, a pair of primers with NheI/BamHI recognition sites was designed. The exact insertion of the 3D8 VL 4M gene into pCTCON was identified by base sequencing analyses, followed by the transformation of the recombinant vector into Saccharomyces cerevisiae EBY100. Transformed colonies were cultured at 30° C. for 20 hrs in selective SD-CAA media (20 g/L glucose, 6.7 g/L yeast nitrogen base without amino acids, 5.4 g/L Na₂HPO₄, 8.6 g/L NaH₂PO₄.H₂O, 5 g/L casamino acids) with agitation. Protein expression was achieved by incubation at 30° C. for 20 hrs in SG-CAA media (20 g/L galactose, 6.7 g/L yeast nitrogen base without amino acids, 5.4 g/L Na₂HPO₄, 8.6 g/L NaH₂PO₄.H₂O, 5 g/L casamino acids). The cell-surface expression of the desired protein was determined using FACS. The expression of 3D8 VL 4M on yeast cell surfaces could be identified by detecting a C-terminal myc-tag. This expression was analyzed qualitatively and quantitatively by using an anti-myc 9E10 antibody (Sigma, USA) as a primary antibody with FITC conjugated-goat anti-mouse IgG (Sigma, USA) serving as a secondary antibody for recognizing the constant region of the primary antibody. In order to determine the cell-surface expression and target substrate binding levels of the 3D8 VL library, about 2×10⁶ yeast cells were treated with biotin-labeled nucleic acids in 50 μl of Tris buffer (25 mM Tris, 137 mM NaCl, 2.7 mM KCl, 0.1% BSA) and then with an anti-myc antibody, washed with Tris buffer and labeled with an FITC conjugated-goat anti-mouse IgG. Quantitative analysis performed on BD FACS Calibur (Becton Dickinson, USA) showed high expression levels of 3D8 VL 4M on yeast cell surfaces.

2. Selection of Model Sequence

A model sequence for modifying the 3D8 VL 4M antibody into a variant with sequence specificity was selected. The biotinylated nucleic acid used in 1 was used as a model sequence. The corresponding sequence was Human epidermal growth factor-2 (Her2/ErbB2) which is known to be overexpressed in breast carcinoma cells and thus involved in the growth and metastasis of cancer cells. Among the entire sequence of Her2, only 18 residues, corresponding to positions 2391-2408, identified as 5′-AAT TCC AGT GGC CAT CAA-3′, were used for the antibody engineering, and called Her2₁₈. Another model sequence, called G₁₈, identified as 5′-GGG GGG GGG GGG GGG GGG-3′ was also used for model target substrates.

3D8 VL WT and 3D8 VL 4M have amino acid sequences of SEQ ID NOS: 1 and 2, respectively. Their corresponding base sequences are represented by SEQ ID NOS: 3 and 4, respectively.

### Example 2

### Construction of 3D8 VL 4M Antibody Library

After 3D8 VL 4M was observed to be expressed at a high level on yeast cell surfaces, a 3D8 VL 4M library was constructed. For the generation of variants which bind specifically to and hydrolyze certain base sequences, libraries were constructed based on the template of 3D8 VL 4M. First, the structure of 3D8 VL was analyzed to determine a putative nucleic acid-binding site composed of the c-, c′- and f-β-strands. It was designed to randomize targeted mutation residues at the in c- (residues 41-45), c′- (residues 50-54) and f-β-strand (residues 90-94) with degenerate NNB codons (N=A/T/C/G, B=C/G/T) to generate library on yeast cell surfaces. Because 3D8 VL was not mutated at all residues, the yeast surface-displayed gene libraries were constructed on the template of 4M using overlapping PCR mutagenesis with primers which had mutations at certain residues. The base sequences of the primers (1F, 2R, 3R, 4F, 5R, 6F, 7R) used for the library construction are given as SEQ ID NOS: 5 to 11, respectively. In the NNB codon, N stands for an equimolar nucleotide mixture of A, T, C and G (25% each), and B for an equimolar nucleotide mixture of C, G and T (33% each). The NNB codon is a combination of codons for all 20 amino acids with a stop codon rate of 2.1%.

The amplified library were transformed, together with a yeast surface-display vector, into yeast cells by homologous recombination. For this, the amplified gene libraries (10 μg/ml) and a yeast surface-display vector (pCTCON, Colby et al., Methods enzymol, 388:248-258) (1 μg/ml) were introduced into yeast cells using an electroporation technique to display the libraries on the yeast cell surface (Lee H W et al., Biochem Biophys Res Commun, 343:896-903, 2006; Kim Y S et al., Proteins: structure, function, and bioinformatics, 62:1026-1035, 2006). The library gene was prepared in a total amount of 300 μg while the vector was used in an amount of 30 μg. 3D8 VL 4M library size determined by plating serial dilutions of the transformed cell on the selective agar plates was about 2×10⁸.

The expression of the library was quantitatively analyzed using FACS. Because any problem occurred during the construction did not permit the normal expression of the library gene, FACS analysis also made it possible to examine whether the library was constructed well.

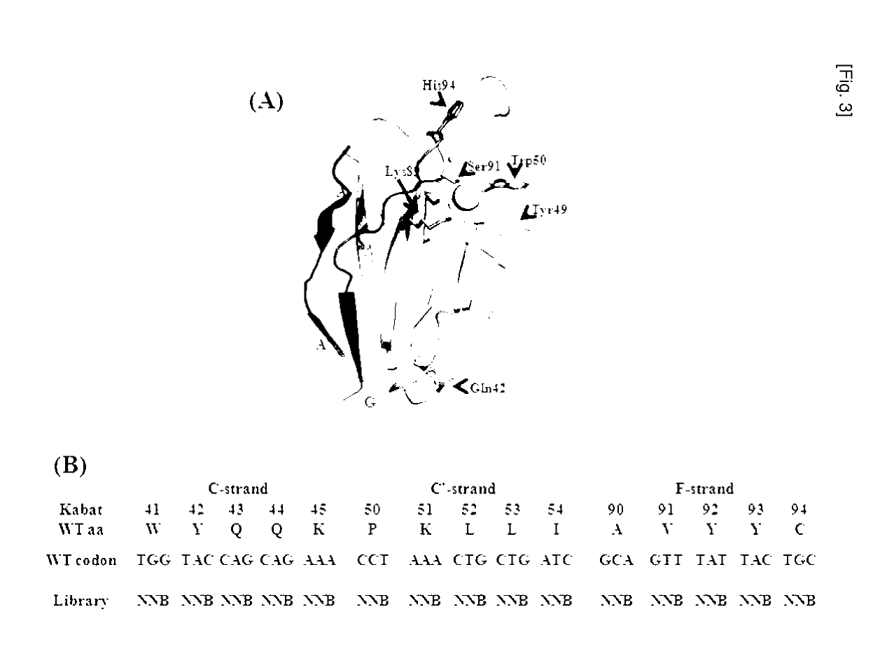

FIG. 3 shows the tertiary structure of 3D8 VL (A) and the amino acid sequences and base sequences of the c- (residues 41-45), c′- (residues 50-54) and f-β-strand (residue 90-94) constituting the putative DNA/RNA recognition site of 3D8 VL WT, and the NNB codons used for mutation (B).

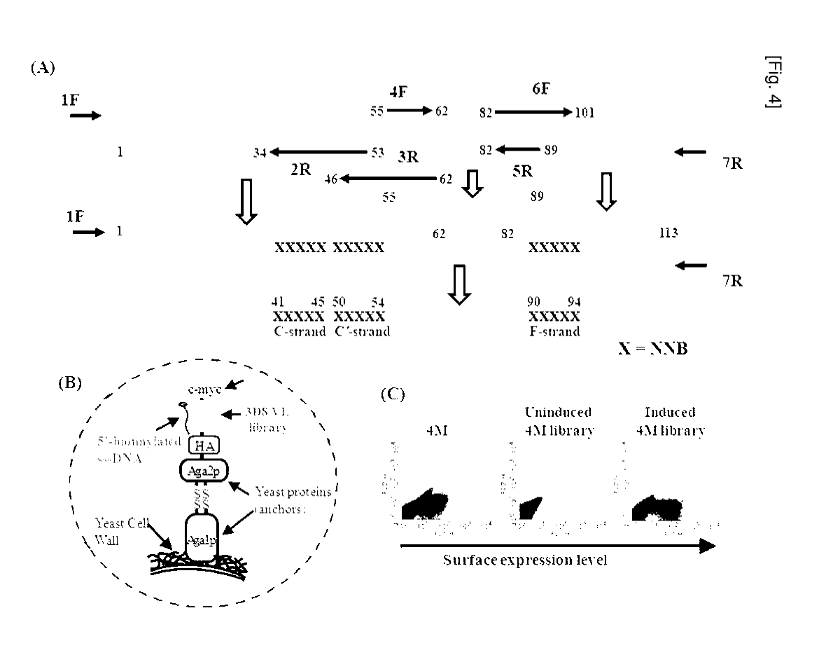

With reference to FIG. 4, there are schematic views showing the construction of a library of nucleic acid-hydrolyzing antibody on the template of 3D8 VL 4M (A), the expression of the library on yeast cell surfaces following cotransformation with a yeast display vector (pCTCON) by electroporation (B) and FACS analysis of the expression levels of the library (C).

Frequencies of mutants in the constructed libraries are given in Table 1, below.

TABLE 1
Library(NNB codon)

| amino acid | frequency | percentage(%) |
|---|---|---|
| Phe | 2 | 4.2% |
| Leu | 4 | 8.3% |
| Ile | 2 | 4.2% |
| Met | 1 | 2.1% |
| Val | 3 | 6.3% |
| Ser | 5 | 10.4% |
| Pro | 3 | 6.3% |
| Thr | 3 | 6.3% |
| Ala | 3 | 6.3% |
| Tyr | 2 | 4.2% |
| His | 2 | 4.2% |
| Gln | 1 | 2.1% |
| Asn | 2 | 4.2% |
| Lys | 1 | 2.1% |
| Asp | 2 | 4.2% |
| Glu | 1 | 2.1% |
| Cys | 2 | 4.2% |
| Trp | 1 | 2.1% |
| Arg | 4 | 8.3% |
| Gly | 3 | 6.3% |
| Stop | 1 | 2.1% |
| Sum | 48 | — |

As seen in FIG. 4, the libraries constructed on the template of 3D8 VL 4M were normally expressed, demonstrating that the libraries were displayed on yeast cell surface well.

### Example 3

### Selection of Variants Specific for Target Sequence from Libraries of 3D8 VL 4M

1. Screening of Libraries of 3D8 VL 4M Using Competitor

The constructed libraries were screened against two types of 5′-biotinylated DNA using MACS and FACS. The MACS and FACS screening was performed at a high salt concentration (0.3M) to exlude non-specific binders that interacts with DNA phosphate backbone through electrostatic interactions. To ensure that selected 3D8 VL variants will bind specifically to the given target sequences, non-biotinylated off-target competitors (DNA) was added to the target substrate. N₁₈ DNA was used as a competitor for Her2₁₈. In order to detect the clones selectively binding to G₁₈, three types of DNA, A₁₈, T₁₈ and C₁₈ were used as competitors at a NaCl concentration of 0.3 M. Base sequences of the 5′-biotinylated substrates (G₁₈, Her2₁₈) used for screening variants specific for target base sequences are represented by SEQ ID NOS: 12 and 13, respectively.

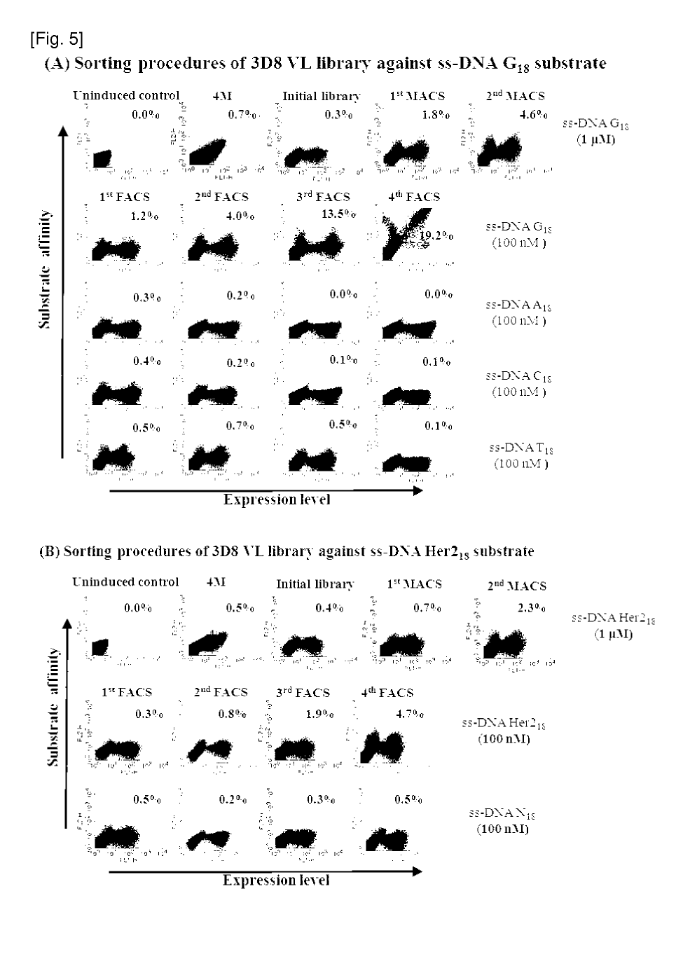

FIG. 5 shows the representative Screening procedures for the isolation of 3D8 VL variants preferentially binding to the two ss-DNA target substrates, G₁₈ (A) and Her2₁₈ (B), from the yeast surface-displayed 3D8 VL library.

With the increase in screening round, as seen in FIG. 5, the variants specifically binding to target sequences were enriched. The variants enriched an each round of the screening were found to have high affinity for target sequences, but low affinity for off-targets.

2. Analysis of High Affinity Variants for Binding Specificity

After the FACS analysis of variants for binding to targets (G₁₈, Her2₁₈) and off-targets, 11 variants were selected against the single-stranded DNA targets (G₁₈, Her2₁₈): 4MG1, 4MG2, 4MG3, 4MG4, 4MG5 and 4MG6 against the single-stranded DNA target G₁₈, and 4MH1, 4MH2, 4MH3, 4MH4 and 4MH5 against the single-stranded DNA target Her2₁₈. These 11 variants are represented by SEQ ID NOS: 14 to 24 with respect to the amino acid sequences thereof, respectively, with the base sequences of SEQ ID NOS: 25 to 35 corresponding thereto.

The selected 11 variants were analyzed for binding specificity for the target substrates (G₁₈, Her2₁₈) and off-targets by FACS. Coincident with the data of the library screening, their affinity was measured to be high for their target single-stranded DNA substrates (G₁₈, Her2₁₈), but relatively low for off-targets.

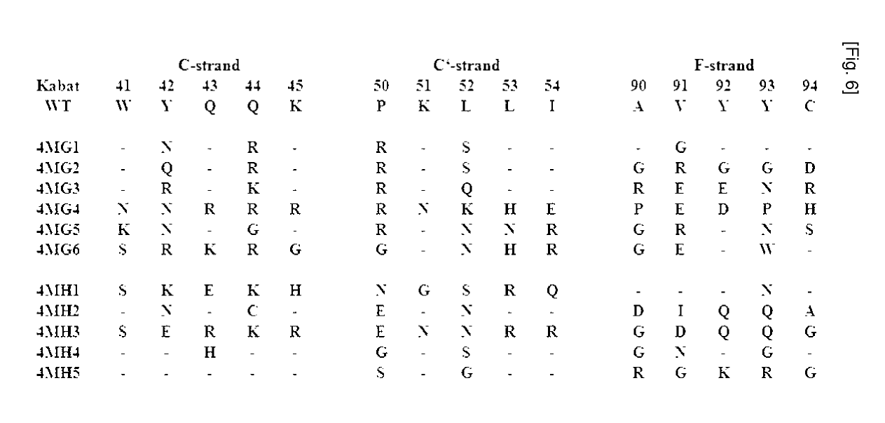

In order to examine the sequences of the 11 variants, plasmids carrying the variants were subjected to base sequencing analyses following purification and amplification. Referring to FIG. 6, the 11 variants are shown for their amino acid sequences of the c- (residues 41-45), c′- (residues 50-54) and f-β-strand (residues 90-94).

### Example 4

### Expression, Purification, and HLPC and CD Analysis of Selected Variants

In order to purify the selected variants of Example 3 in soluble forms, an examination was first made of the expression of them with yeast and E. coli expression vectors. Because of high expression levels of the variants on yeast cell surfaces, they were first subcloned in-frame into yeast expression vectors which were in turn transformed into a Saccharomyces cerevisiae 2805 strain. In contrast to the surface expression, they were not expressed solubly well in the yeast strain. Thus, an E. coli BL21(DE3) strain was employed as an expression system. Although expressed at a high level in E. coli, the selected variants were not purified in a soluble fraction. Almost all of the variants were expressed dominantly in an insoluble form of inclusion body. Thus, the proteins in the form of inclusion body were purified and refolded (Lee S H et al., Protein Science, 15:304-313, 2006).

The purity of the purified 11 variants was determined by SDS-PAGE while HPLC was performed to examine whether the variants, after purification from the inclusion body, existed solubly as monomers. In addition, the antibody libraries according to the present invention were designed to have mutations in the framework, but not in the CDR, unlike typical antibody libraries. Thus, the selected variants were examined for secondary structure using Far-UV CD (circular dichroism) spectroscopy.

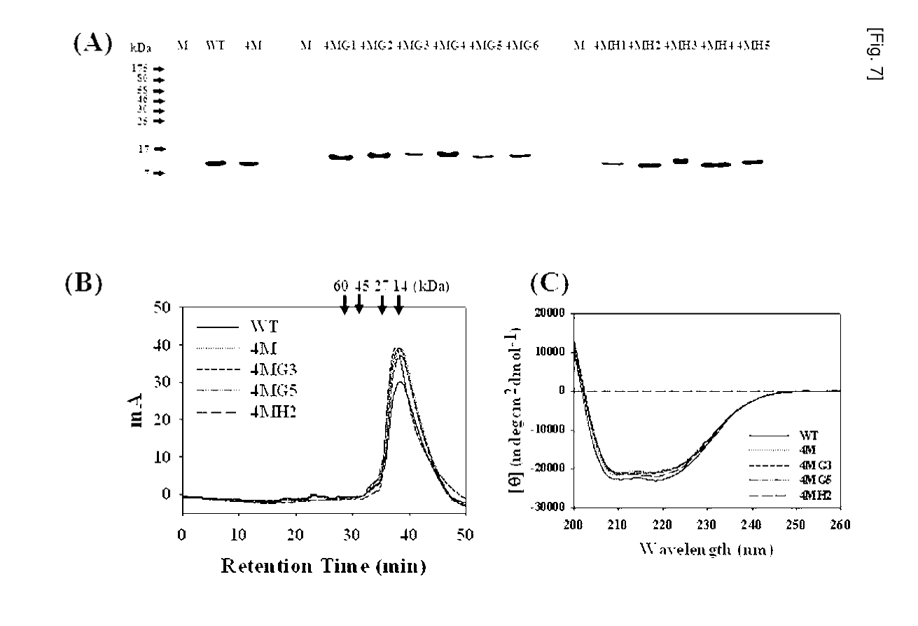

With reference to FIG. 7, data for the purified 11 variants are given of SDS-PAGE (A), HPLC (B) and Far-UV CD spectroscopy (C).

As seen in FIG. 7, all of the 11 variants were found to be greater than 90% in purity as measured by SDS-PAGE (A). Main HPLC peaks of the variants (4MG3, 4MG5 and 4MH2) were read at the same positions as in 3D8 VL WT and 3D8 VL 4M, demonstrating that most of the purified proteins existed in a soluble form of monomers (B). As for the secondary structure, the variants 4MG3, 4MG5 and 4MH2 exhibited Far-UV CD spectra very similar to those of 3D8 VL WT and 3D8 VL 4M.

### Example 5

### Affinity for Nucleic Acid and Nucleic Acid-Hydrolyzing Activity of Selected Variants

1. Affinity of Variants for Nucleic Acid

The selected variants were subjected to SPR analysis using Biacore2000. The specificity and affinity of the selected variants and 3D8 VL WT for the target (G₁₈) and off-targets were evaluated.

The results are summarized in Table 2, below.

TABLE 2

| | Kinetic | ss-DNA substrates | | | | | |
|---|---|---|---|---|---|---|---|
| Proteins | Parameters | A₁₈ | T₁₈ | C₁₈ | G₁₈ | Her2₁₈ | N₁₈ |
| WT | kₒₙ(M⁻¹s⁻¹)(×10³) | 0.13 ± 0.04 | 0.67 ± 0.02 | 0.78 ± 0.04 | 0.71 ± 0.01 | 1.63 ± 0.30 | 4.11 ± 0.33 |
| | k_{off} (s⁻¹)(×10⁻³) | 3.24 ± 0.21 | 6.82 ± 0.13 | 9.73 ± 0.88 | 8.26 ± 0.62 | 35.2 ± 2.3 | 15.4 ± 2.7 |
| | K_{D} (M) (×10⁻⁷) | 23.9 ± 1.3 | 10.2 ± 3.0 | 12.6 ± 3.3 | 12.6 ± 1.5 | 21.5 ± 5.3 | 37.5 ± 4.2 |
| 4M | kₒₙ(M⁻¹s⁻¹)(×10³) | 0.60 ± 0.02 | 3.95 ± 0.21 | 5.81 ± 0.25 | 0.67 ± 0.03 | 0.24 ± 0.02 | 0.96 ± 0.02 |
| | k_{off} (s⁻¹)(×10⁻³) | 7.32 ± 0.70 | 5.80 ± 0.09 | 5.88 ± 0.71 | 1.18 ± 0.10 | 5.49 ± 0.47 | 10.3 ± 1.8 |
| | K_{D} (M) (×10⁻⁷) | 12.2 ± 1.2 | 14.7 ± 4.0 | 30.1 ± 4.6 | 17.2 ± 5.3 | 23.3 ± 2.7 | 10.7 ± 3.5 |
| 4MG3 | kₒₙ(M⁻¹s⁻¹)(×10³) | 0.14 ± 0.03 | 0.25 ± 0.07 | 3.07 ± 0.32 | 1.17 ± 0.3 | 0.24 ± 0.04 | 0.38 ± 0.02 |
| | k_{off} (s⁻¹)(×10⁻³) | 5.24 ± 0.95 | 2.80 ± 0.19 | 3.53 ± 0.41 | 0.09 ± 0.01 | 7.55 ± 0.89 | 9.37 ± 0.97 |
| | K_{D} (M) (×10⁻⁷) | 37.1 ± 2.8 | 10.0 ± 2.2 | 11.5 ± 1.4 | 0.76 ± 0.04 | 31.3 ± 2.4 | 24.9 ± 3.1 |
| 4MG5 | kₒₙ(M⁻¹s⁻¹)(×10³) | 0.26 ± 0.07 | 0.89 ± 0.04 | 0.51 ± 0.03 | 9.02 ± 0.77 | 1.25 ± 0.52 | 0.13 ± 0.03 |
| | k_{off} (s⁻¹)(×10⁻³) | 79.2 ± 2.3 | 31.3 ± 2.5 | 1.92 ± 0.51 | 0.84 ± 0.49 | 5.32 ± 0.82 | 7.25 ± 0.87 |
| | K_{D} (M) (×10⁻⁷) | 30.3 ± 1.9 | 35.0 ± 3.6 | 37.7 ± 2.9 | 0.93 ± 0.03 | 42.1 ± 2.9 | 54.7 ± 5.4 |
| 4MH2 | kₒₙ(M⁻¹s⁻¹)(×10³) | 0.13 ± 0.01 | 0.25 ± 0.05 | 0.43 ± 0.03 | 0.42 ± 0.07 | 4.41 ± 0.13 | 0.17 ± 0.01 |
| | k_{off} (s⁻¹)(×10⁻³) | 2.54 ± 0.33 | 2.51 ± 0.61 | 4.31 ± 0.87 | 4.53 ± 0.82 | 0.94 ± 0.07 | 7.20 ± 0.66 |
| | K_{D} (M) (×10⁻⁷) | 19.4 ± 5.1 | 10.3 ± 3.7 | 14.0 ± 2.2 | 10.7 ± 3.4 | 2.13 ± 0.13 | 42.7 ± 2.9 |

As seen in Table 2, the variants show a great difference in affinity between the targets and the off-targets whereas WT and 4M do not significantly differ in affinity from one sequence to another sequence. The variants selected from the libraries constructed on the template of 3D8 VL 4M were greatly improved in affinity for the targets Her2₁₈ and G₁₈, but remained unchanged in affinity for off-targets, with about 10˜100-fold difference in affinity between them, which demonstrated that the variants of the present invention was modified to bind specifically to the targets.

2. Nucleic Acid-Hydrolyzing Activity of Variants

The nucleic acid-hydrolyzing activity of the purified variants was assayed with agarose gel electrophoresis. A pUC19 plasmid was used as a substrate. It was purified with the aid of a miniprep kit (Intron Inc., Korea). Greater than 95% of the purified pUC19 plasmid was in the form of supercoiled plasmid as patterned on 0.7% agarose gel. A hydrolytic reaction between the plasmid substrate and the variants was conducted in TBS (Tris buffered saline) containing 2 mM MgCl₂ or 50 mM EDTA. In all hydrolysis reactions, ionic strength was fixed at 150 mM with the NaCl of TBS. The antibody was incubated with the nucleic acid at 37° C. for 1 hr. After the incubation, the reaction mixture was treated at 37° C. for 1 hr with trypsin protease (20 μg/ml) (Sigma, USA) to prevent the phenomenon that the 3D8 antibody-bound nucleic acid remained at an upper position upon agarose gel electrophoresis. Following electrophoresis on 0.7% agarose gel, the samples were stained with ethidium bromide.

Also, some of the variants were examined for RNA hydrolyzing activity. The three variants 4MG3, 4MG5 and 4MH2 were subjected, together with 3D8 VL WT and 3D8 VL 3M, to RNA hydrolysis, with RNase A and HW1 serving as controls. As will be demonstrated later, these variants showed good performance on sequence-specific hydrolysis. RNA hydrolysis was performed in TBS containing 2 mM MgCl₂ or 50 mM EDTA using the total RNA isolated from HeLa cells.

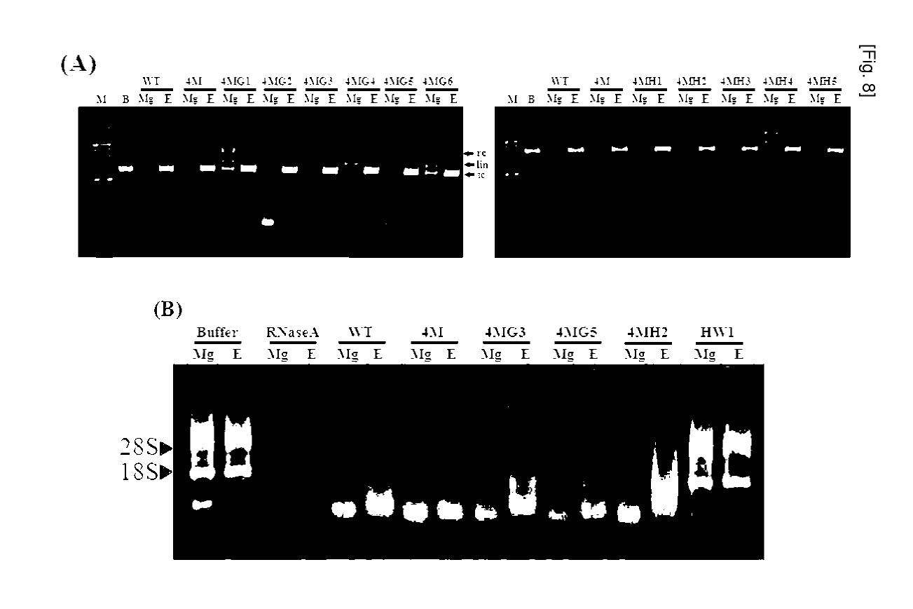

FIG. 8 shows results of the agarose gel electrophoresis for DNA-hydrolyzing activity of the 11 variants (A) and for RNA-hydrolyzing activity of 4MG3, 4MG5 and 4MH2 (B).

As seen in FIG. 8, seven (4MG2, 4MG3, 4MG5, 4MH1, 4MH2, 4MH3 and 4MH5) of the 11 variants were found to have DNA-hydrolyzing activity and the remaining four (4MG1, 4MG4, 4MG6, and 4MH4) were significantly lower in hydrolyzing activity, compared to 3D8 VL 4M (A). RNase A hydrolyzed almost all RNAs while HW1 could not, like the buffer control. On the other hand, the variants exhibited RNA-hydrolyzing activity even in the present of EDTA, like WT, 4M and RNase A (B).

Therefore, the variants according to the present invention can hydrolyze both DNA and RNA in vitro.

### Example 6

### Sequence-Specific, Nucleic Acid-Hydrolyzing Activity of Selected Variants

The variants proven for nucleic acid-hydrolyzing activity were examined for sequence specificity in accordance with the purpose of the present invention. The purified variants were incubated with fluorescence-labeled primers, followed by the analysis of fluorescent signals using a FRET (fluorescence resonance energy transfer)-based cleavage assay. The primers were double-labeled with the green fluorescent 6-FAM at 5′-terminus and its quencher BHQ-1 at 3′-terminus. When the primers remained unhydrolyzed, no fluorescence signals were detected because the fluorescence of 6-FAM was absorbed by the adjacent BHQ-1. On the other hand, when the primers were hydrolyzed at residues between the 5′- and the 3′-end by the variants, the fluorescence signals of 6-FAM could be read because 6-FAM became distant from BHQ-1. In this regard, the primers A₁₈, T₁₈, C₁₈, Her2₁₈, and N₁₈, used for library screening, were labeled at respective ends with 6-FAM and BHQ-1. As for G₁₈, it was substituted with the primer (G₄T)₃G₃ in which a set of 4 guanine residues and one thymine residue was arrayed in tandem because it was difficult to synthesize. The base sequences of the FRET substrates (A₁₈, T₁₈, C₁₈, (G₄T)₃G₃, Her2₁₈, N₁₈) used in assay for sequence-specific, nucleic acid-hydrolyzing activity are represented by SEQ ID NOS: 36 to 41, respectively.

The three variants 4MG3, 4MG5 and 4MH2 were found to have sequence-specific, nucleic acid-hydrolyzing activity as measured by FRET assay. In order to obtain more exact enzyme kinetic parameters, the antibodies at a fixed concentration of 100 nM were incubated with the substrates at various concentrations of from 16 nM to 2 μM during which the dissociation constants of antibodies were measured at each substrate concentration. On the whole, the reaction rate of an enzyme increases with increasing of substrate concentration if other conditions are fixed, but does not significantly increase as it approaches near Vmax.

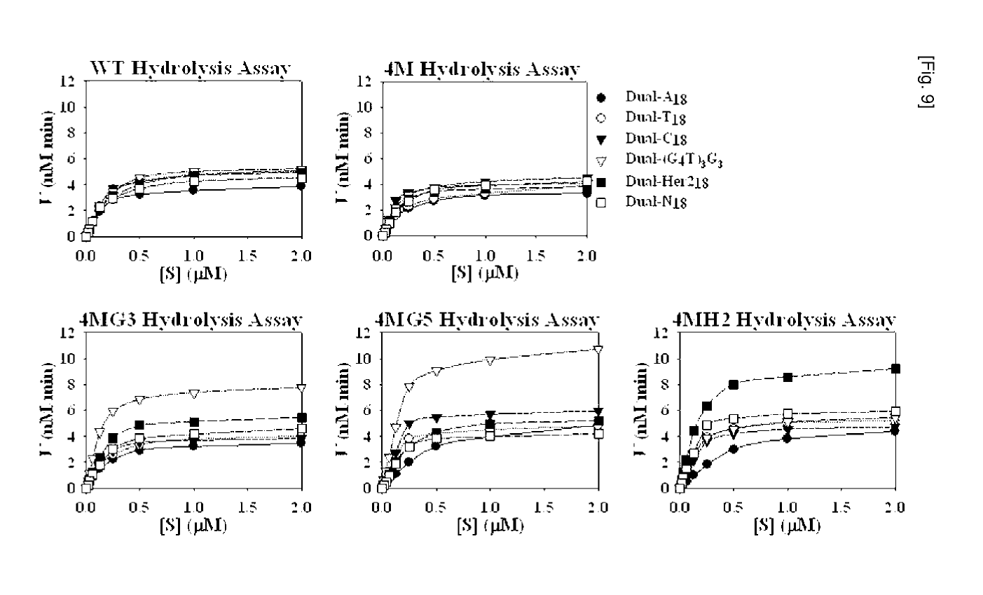

The antibodies 3D8 VL WT and 3D8 VL 4M and the variants (4MG3, 4MG5, 4MH2) were measured for enzyme kinetics while the FRET substrates (A₁₈, T₁₈, C₁₈, (G₄T)₃G₃, Her2₁₈, N₁₈) varied in concentration from 16 nM to 2 μM, and the results are depicted in FIG. 9. Km, Kcat, Kcat/Km values of the antibodies were calculated from the FRET data and given in Table 3, below.

TABLE 3

| | | | | | Dual- | | |
|---|---|---|---|---|---|---|---|
| | | Dual-A₁₈ | Dual-T₁₈ | Dual-C₁₈ | (G₄T)₃G₃ | Dual-Her2₁₈ | Dual-N₁₈ |
| WT | Kₘ(nM) | 642 | 563 | 520 | 549 | 534 | 578 |
| | k_{cat}(s⁻¹)(×10⁻³) | 1.61 | 1.79 | 1.69 | 1.80 | 1.63 | 1.71 |
| | Vₘₐₓ (nM s⁻¹)(×10⁻¹) | 1.61 | 1.79 | 1.69 | 1.80 | 1.63 | 1.71 |
| | k_{cat}/Kₘ (nM⁻¹s⁻¹) (×10⁻⁶) | 2.51 | 3.17 | 3.24 | 3.28 | 3.05 | 2.96 |
| 4M | Kₘ(nM) | 609 | 560 | 604 | 521 | 583 | 573 |
| | k_{cat}(s⁻¹)(×10⁻³) | 1.27 | 1.22 | 1.93 | 1.55 | 1.46 | 1.51 |
| | Vₘₐₓ (nM s⁻¹)(×10⁻¹) | 1.27 | 1.22 | 1.93 | 1.55 | 1.46 | 1.51 |
| | k_{cat}/Kₘ (nM⁻¹s⁻¹) (×10⁻⁶) | 2.08 | 2.18 | 3.19 | 2.97 | 2.50 | 2.62 |
| 4MG3 | Kₘ(nM) | 685 | 830 | 510 | 307 | 491 | 549 |
| | k_{cat}(s⁻¹)(×10⁻³) | 1.43 | 1.90 | 1.33 | 2.08 | 1.71 | 1.51 |
| | Vₘₐₓ (nM s⁻¹)(×10⁻¹) | 1.43 | 1.90 | 1.33 | 2.08 | 1.71 | 1.51 |
| | k_{cat}/Kₘ (nM⁻¹s⁻¹) (×10⁻⁶) | 2.08 | 2.29 | 2.61 | 6.75 | 3.47 | 2.75 |
| 4MG5 | Kₘ(nM) | 828 | 495 | 521 | 362 | 640 | 589 |
| | k_{cat}(s⁻¹)(×10⁻³) | 1.41 | 1.68 | 1.97 | 2.72 | 1.79 | 1.55 |
| | Vₘₐₓ (nM s⁻¹)(×10⁻¹) | 1.41 | 1.68 | 1.97 | 2.72 | 1.79 | 1.55 |
| | k_{cat}/Kₘ (nM⁻¹s⁻¹) (×10⁻⁶) | 1.70 | 3.39 | 3.69 | 7.51 | 2.70 | 2.63 |
| 4MH2 | Kₘ(nM) | 744 | 546 | 518 | 472 | 305 | 438 |
| | k_{cat}(s⁻¹)(×10⁻³) | 1.14 | 1.84 | 1.65 | 1.76 | 2.16 | 1.88 |
| | Vₘₐₓ (nM s⁻¹)(×10⁻¹) | 1.14 | 1.84 | 1.65 | 1.76 | 2.16 | 1.88 |
| | k_{cat}/Kₘ (nM⁻¹s⁻¹) (×10⁻⁶) | 1.53 | 3.37 | 3.17 | 3.72 | 7.06 | 4.30 |

As seen in FIG. 9 and Table 3, the variants (4MG3, 4MG5, 4MH2) had much higher Vmax with regard to respective target substrates, compared to 3D8 VL WT and 3D8 VL 4M, indicating that when sufficient substrates are present, the variants can hydrolyze target substrates faster than other substrates. In contrast, the reaction rates of 3D8 VL WT and 3D8 VL 4M were almost independent of substrate sequences. The kinetic parameters Km, Kcat, and Kcat/Km of the antibodies were calculated from the obtained results. Of the three variants, 4MG3 and 4MG5 hydrolyzed the target G₁₈ with high sequence specific as demonstrated by their higher Vmax for the target G₁₈ than off-targets. As for the 4MH2 variant, its Vmax was higher for the target Her2₁₈ than off-targets, accounting for the specific recognition and hydrolysis of Her2₁₈ thereby. These variants exhibited lower Km and higher Kcat/Km for target substrates than off-targets. Km means a half of the substrate concentration at which an antibody reach Vmax. Thus, the smaller the Km is, the higher the antibody is in affinity for substrate. The variants 4MG3, 4MG5 and 4MH2 had smaller Km values for respective target substrates than other substrates. A difference between the Km and the Kd measured using Biacore2000 is thought to be attributed to the fact that Km does not account for affinity only. The Kcat/Km of the variants was two to five-fold higher for target substrates than off-targets. Higher Kcat/Km values mean more potent hydrolyzing activity for a substrate.

Consequently, the variants 4MG3, 4MG5 and 4MH2 can specifically recognize and hydrolyze respective target sequences faster than off-targets.

### Example 7

### Sequence-Specific, Nucleic Acid-Hydrolyzing Activity of the Variants within Cells

A reporter system with a green fluorescent EGFP gene was employed to evaluate the cytosolic, sequence-specific, nucleic acid-hydrolyzing activity of the variants. The synthetic target sequences G₁₈ and Her2₁₈ were placed between the ATG start codon and the EGFP coding sequence in pEGFP-N1 plasmid to afford pEGFP-N1-G₁₈ and pEGFP-N-1-Her2₁₈, respectively. For use in transfection into mammal cells, 3D8 VL WT and the variants (4MG3, 4MG5, 4MH2) were subcloned to respective expression vector pcDNA3.1 (+). In greater detail, HeLa cells were plated at a density of 2×10⁵ cells/well in 6-well plates containing 2 ml of DMEM supplemented with 10% FBS and incubated at 37° C. for 24 hrs in a 5% CO₂ atmosphere. Once the cells were stabilized, the medium was removed and each well was washed with 1 ml of PBS. Then, 800 μl of TOM (Transfection optimized medium, WelGENE Inc., Korea) was added to each well to obtain maximum efficiency for transfection. 500 ng of pEGFP-N1 alone 500 ng of pEGFP-N1-G₁₈ alone 500 ng of pEGFP-N1-Her₁₈ alone 500 ng of pEGFP-N1 in combination of 500 ng of pcDNA3.1(+)-wild type, pcDNA3.1(+)-4MG3, pcDNA3.1(+)-4MG5 or pcDNA3.1(+)-4MH2; 500 ng of pEGFP-G₁₈ in combination of 500 ng of pcDNA3.1(+)-wild type, pcDNA3.1(+)-4MG3, or pcDNA3.1(+)-4MG5; or 500 ng of pEGFP-Her2₁₈ in combination with 500 ng of pcDNA3.1(+)-wild type or pcDNA3.1(+)-4MH2 were reacted at room temperature for 20 min with 5 μl of Lipofectamine 2000 (Invitrogen, USA) in 200 μl of TOM medium and added to each well. Following incubation at 37° C. for 6 hrs in a 5% CO₂, the TOM medium was changed with 2 ml of 10% FBS-supplemented DMEM. 24 Hours post transfection, the medium was removed and cells were obtained with trypsin-EDTA and washed with PBS. GFP fluorescence was measured from each sample using FACS Caliber (Fluorescent Activated Cell Sorter).

Each transfected sample was treated with rabbit anti-3D8 polyclonal antibody and subsequently with a TRITC-conjugated anti-rabbit antibody to stain 3D8 VL wild-type, 4MG3, 4MG5 and 4MH2. Nuclei were stained with DAPI. A confocal microscope was used to determine the expression levels of EGFP (green), and 3D8 VL wild-type, 4MG3, 4MG5 and 4MH2 proteins (red).

Proteins and total RNAs were isolated from each transfected sample and subjected to Western blotting and RT-PCR, respectively, to examine the EGFP reduction by 3D8 VL wild-type and the variants (4MG3, 4MG5, 4MH2) at protein and mRNA levels.

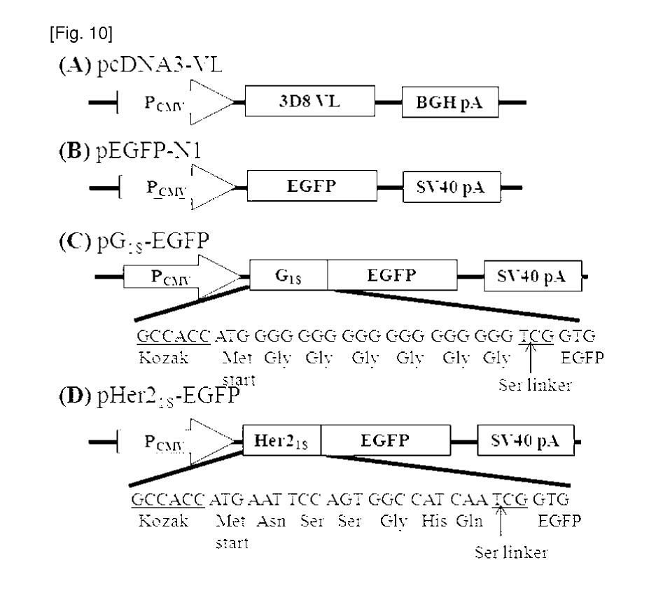

With reference to FIG. 10, plasmids for the cytosolic expression of 3D8 VL wild-type and the variants (4MG3, 4MG5) (A, pcDNA3.1), GFP (B, pEGFP-N1), GFP (C, pG₁₈-EGFP in which G₁₈ is located in the N-terminal upstream of EGFP), and EGFP (D, pHer2₁₈-EGFP in which Her2₁₈ is located in the N-terminal upstream of EGFP) are shown.

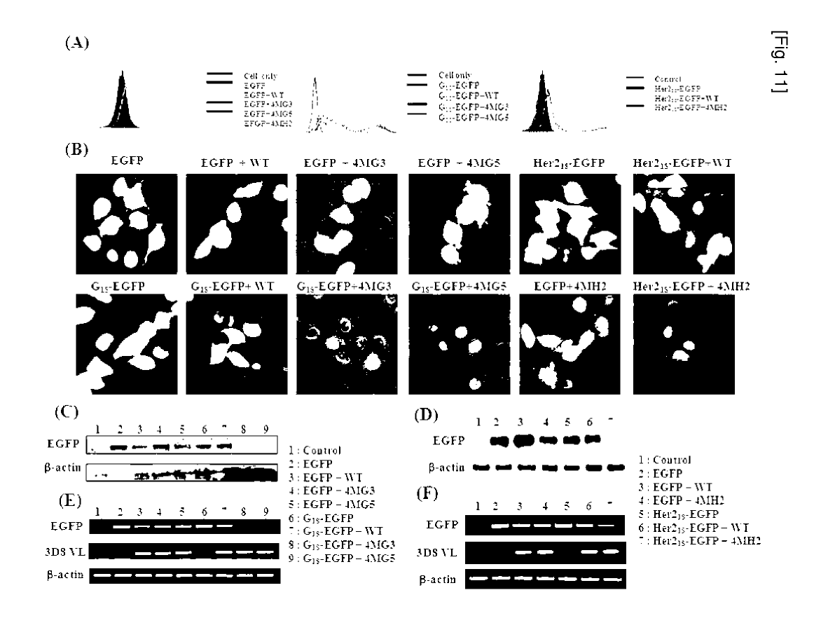

FIG. 11 shows the expression levels of reporter EGFP containing the target base sequence by 3D8 VL variants when 3D8 VL wild-type or the variants (4MG3, 4MG5, 4MH2), and various EGFP reporter gene (EGFP, G₁₈-EGFP, Her2₁₈-EGFP) were transfected by the expression vectors of FIG. 10 in HeLa cells, in terms of fluorescence level through FACS (A), confocal microscopy (B), Western blotting (C, D) and RT-PCR (E, F).

As seen in FIG. 11A, the expression level of EGFP did not significantly differ from 3D8 VL wild-type to the variants (4MG3, 4MG5, 4MH2) in the absence of G₁₈ and Her2₁₈. On the other hand, when G₁₈ was located before the 5′-terminus of EGFP, the fluorescence signal of EGFP was detected at a far lower level in the cells expressing the variants (4MG3, 4MG5) than the cells expressing 3D8 VL wild-type. Likewise, cells produced far weaker EGFP signals when they were transfected with a vector in which Her2₁₈ was located before the 5′-terminus of EGFP, along with a vector carrying 4MH2, rather than along with a vector carrying 3D8 VL wild-type. Accordingly, when expressed in the cytosol, the variants 4MG3 and 4MG5 hydrolyze G₁₈-EGFP mRNA, which contains the target sequence thereof and the variant 4MH2 catalytically acts on Her2₁₈-EGFP mRNA which contains the target sequence thereof, thus reducing the expression levels of the proteins encoded by the mRNAs.

Also, the same results as in FIG. 11A are given in terms of confocal microscopic data in FIG. 11B. In the absence of G₁₈ and Her2₁₈, no significant differences in EGFP expression level were found between cells expressing 3D8 VL wild-type and cells expressing 4MG3, 4MG5 and 4MH2. In contrast, cells transfected with a vector in which G₁₈ is present before the 5′-terminus of EGFP were found to produce far weaker EGFP fluorescence signals when they expressed 4MG3 or 4MG5, compared to when they expressed 3D8 VL wild-type. Likewise, the cells transfected with a vector in which Her2₁₈ was located before the 5′-terminus of EGFP were measured to produce very lower EGFP fluorescence signals when they expressed 4MH2 than when they expressed VL wild-type. These image results confirmed the data of FIG. 11A, demonstrating that 4MG3, 4MG5, and 4MH2 can recognize respective target sequences and still retain the nucleic acid-hydrolyzing activity.

In FIG. 11C-11F, the cytosolic expression of the variants (4MG3, 4MG5, 4MH2) caused a decrease in the expression level of GFP as identified at both the protein and mRNA levels. Hence, the variants (4MG3, 4MG5, 4MH2) can recognize specific base sequences and hydrolyze them.

### Example 8

### Assay of Her2 Base Sequence Specific, Hydrolyzing Variant (Expression Vector) for Her2 Downregulation

In order to evaluate the Her2 downregulation by the variant 4MH2 containing Her2 base sequence specificity and Her2 hydrolyzing activity, an Her2 gene expression vector was transfected into human cervical carcinoma cells (HeLa), which do not express Her2. Her2 siRNA was used as a positive control for downregulation Her2 mRNA expression. In greater detail, HeLa cells were plated at a density of 2×10⁵ cells/well into 6-well plates containing 2 ml of DMEM supplemented with 10% FBS per well, followed by incubation at 37° C. for 24 hrs in a 5% CO₂ atmosphere. When stabilized, the cells in each well were washed with 1 ml of PBS. Then, 800 μl of TOM (Transfection optimized medium, WelGENE Inc., Korea) was added to each well. 500 ng of pcDNA3.1(+)-Her2 alone was reacted at room temperature for 20 min with 5 μl of Lipofectamine 2000 (Invitrogen, USA) in 200 μl of TOM medium in a tube and added to each well, followed by incubation at 37° C. for 6 hrs in a 5% CO₂ atmosphere. The medium was changed with 2 ml of DMEM supplemented with 10% FBS, and cells were further incubated for 24 hrs. Then, each well was washed with 1 ml of PBS. 800 μl of TOM medium (WelGENE Inc., Korea) was added to each well. After having reacted at room temperature for 20 min with 5 μl of Lipofectamine 2000 (Invitrogen, USA) in 200 μl of TOM medium in a tube, 500 ng of pcDNA3.1(+)-wild type, Her2 siRNA or pcDNA3.1(+)-4MH2 was added to each well. Incubation was conducted at 37° C. for 6 hrs in a 5% CO₂ atmosphere. The medium was exchanged with 2 ml of DMEM supplemented with 10% FBS, followed by incubation for 24 or 48 hrs. After removal of the medium, the cells were obtained by treatment with trypsin-EDTA and washed with PBS. Total RNA and a protein of interest were isolated from each sample and subjected to RT-PCR and Western blotting, respectively, to examine the effects of wild-type, Her2 siRNA, and 4MH2 on Her2 expression at the protein and mRNA levels.

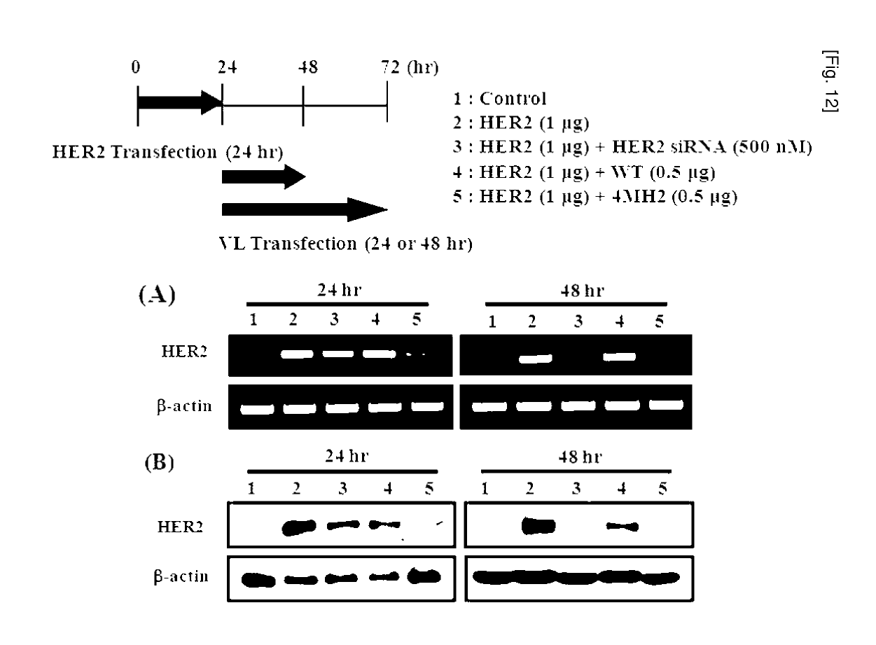

Referring to FIG. 12, the effect of Her2₁₈ base sequence-specific, nucleic acid-hydrolyzing 4MH2 in HeLa cells on Her2 gene expression was analyzed for its mRNA level by RT-PCR (A) and for its protein expression level by Western-blotting (B).

As is apparent from the data of FIG. 12, 4MH2 remarkably downregulated Her2 expression whereas no significant changes were obtained by 3D8 VL wild-type. Particularly, 24 hrs post-transfection, it was observed that 4MH2 caused greater downregulation of Her2 than did Her2 siRNA, indicating that 4MH2 can specifically recognize Her2 sequence and hydrolyze it. Also, 4MH2 was observed to down-regulate both Her2 mRNA and protein to an extent similar to that caused by Her2 siRNA. Particularly, 24 hrs post-transfection, greater downregulation was detected by 4MH2 than Her2 siRNA.

### Example 9

### Cellular Internalization of the Variants (Proteins) and Pathway Thereof

1. Cell-Penetrating Ability of the Variants (Proteins)

3D8 scFV is known to be able to penetrate into cells. FACS and confocal microscopy were used to examine whether 3D8 VL wild-type and variants thereof could penetrate into cells. In detail, HeLa cells were plated at a density of 2×10⁵ cells/well into 6-well plates containing 2 ml of DMEM supplemented with 10% FBS per well and cultured at 37° C. for 24 hrs in a 5% CO₂ atmosphere. When the cells were stabilized, each well was washed with 1 ml of PBS. Then, 800 μl of TOM (Transfection optimized medium, WelGENE Inc., Korea) was added to each well. The cells were treated with the variants (10 μM) before incubation at 37° C. for 2 hrs in a 5% CO₂ atmosphere. After the removal of the medium, the cells were obtained by treatment with trypsin-EDTA and washed with PBS. Each sample was treated with a primary antibody specific for 3D8 scFv and then with a TRITC(red)-labeled secondary antibody to stain 3D8 VL wild-type, 4MG3, 4MG5 and 4MH2. TRITC signals were detected using FACS Calibur (Fluorescent Activated Cell Sorter). At this time, the cells were trypsinized so as to prevent the detection of the proteins which were not internalized into cells but remained attached on the cell surface.

Each transfected sample was treated with a primary antibody specific for 3D8 scFv and then with a TRITC-labeled secondary antibody to stain 3D8 VL wild-type, 4MG3, 4MG5 and 4MH2 proteins. Nuclei were stained with DAPI. A confocal microscope was used to determine the expression levels of EGFP (green fluorescent), and 3D8 VL wild-type, 4MG3, 4MG5 and 4MH2 proteins (red fluorescent).

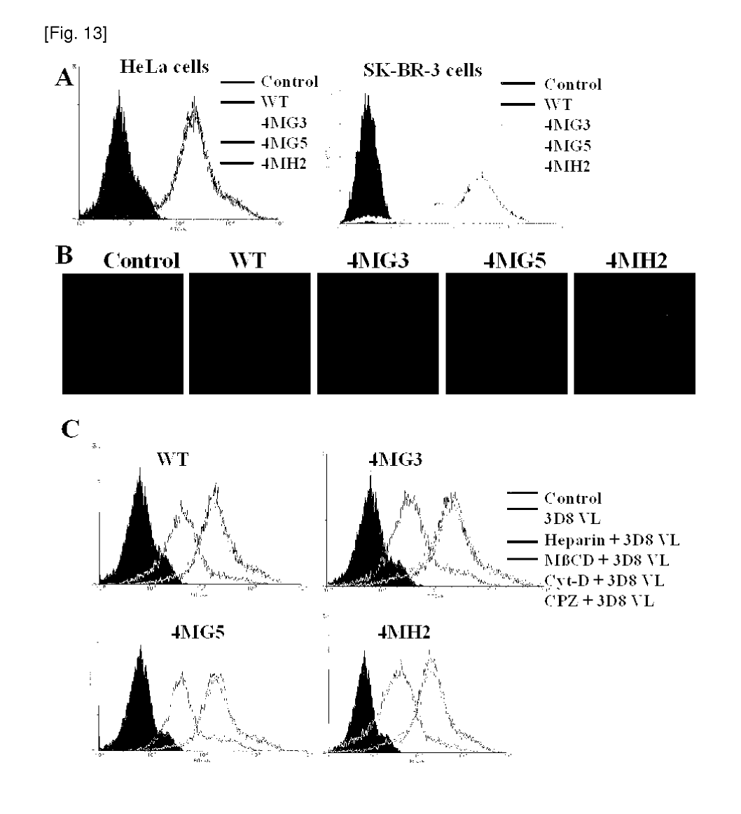

FACS data and confocal microscope data on the internalization of 3D8 VL wild-type and the variants (4MG3, 4MG5, 4MH2) into human cervical carcinoma cells (HeLa) and human breast carcinoma cells (SK-BR3) are given in FIGS. 13A and 13B, respectively.

As shown in FIG. 13A, 3D8 VL wild-type and the variants (4MG3, 4MG5, 4MH2) were observed to penetrate into HeLa and SK-BR-3 to similar extents. In other words, the variants have similar cell-penetrating ability, thus indicating that different in cellular internalization level among the variants does not need to be considered for ongoing or future experiments.

As shown in FIG. 13B, red fluorescent represent 3D8 VL wild-type and the variants (4MG3, 4MG5, 4MH2), and blue fluorescent represent nuleus. Therefore, most of proteins did not penetrate into nuclear membrane, and were translocated into the cytoplasm.

2. Cellular Internalization Pathway of the Variants (Proteins)

To elucidate the specific internalization mechanism of the variants, cells were pretreated with the following pharmacological inhibitors for interfering with the three major endocytic pathways: chlorpromazine (CPZ) for inhibiting clathrin-dependent endocytosis, methyl-β-cyclodextrin (MβCD) for inhibiting caveolae/lipid raft endocytosis, and cytochalasin D (Cyt-D) for inhibiting macropinocycosis. In addition, heparin (100 IU/ml) was also used to interfere with electrical interaction between the positively charged variants and negatively charged proteoglycans (heparan sulfate) on cell surfaces. In greater detail, HeLa cells were plated at a density of 2×10⁵ cells/well into 6-well plates containing 2 ml of DMEM supplemented with 10% FBS per well and cultured at 37° C. for 24 hrs in a 5% CO₂ atmosphere. After the cells were stabilized, each well was washed with 1 ml of PBS. Then, 800 μl of TOM (Transfection optimized medium, WelGENE Inc., Korea) was added to each well. The cells were pre-treated with heparin (5 mM), MβCD (5 mM), chlorpromazine (10 μg/ml), or cytochalasin D (1 μg/ml) for 30 min and then with each variant (10 μM), followed by incubation at 37° C. for 2 hrs in a 5% CO₂ atmosphere. After removal of the medium, the cells were washed with PBS and obtained by treatment with trypsin-EDTA. Each sample was treated with a primary antibody specific for 3D8 scFv and then with a TRITC(red)-labeled secondary antibody to stain 3D8 VL wild-type, 4MG3, 4MG5 and 4MH2. TRITC signals were detected using FACS Calibur (Fluorescent Activated Cell Sorter). As a control of internalization of 3D8 VLs in HeLa cells, without the trearment of soluble heparin or specific endocytosis inhibitors, 3D8 VLs were internalized in HeLa cells and stained with rabbit anti-3D8 polyclonal antibodies and TRITC-labeled anti-rabbit IgG.

FACS data analyzed for effect of pre-treatment of soluble heparin or specific endocytosis inhibitors on the cellular uptakes of 3D8 VL wild-type and the variants (4MG3, 4MG5, 4MH2) are shown in FIG. 13C.

As is apparent from the data of FIG. 13C, the cellular internalization of the variants was not affected by chlorpromazine or cytochalasin whereas pretreatment with heparin or MβCD caused a significant reduction in the cellular internalization of the variants, which demonstrates that the variants primarily electrically interact with cell surface materials such as proteoglycan and then undergo caveolae/lipid raft endocytosis.

### Example 10

### Intracellular Sequence-Specific, Nucleic Acid-Hydrolyzing Activity of the Variants (Proteins)

A reporter gene system was employed to evaluate the cytosolic, sequence-specific, nucleic acid-hydrolyzing activity of the variants. For this, the expression vector pEGFP-N1 carrying an EGFP (green fluorescence) and an expression vector in which 18 guanine residues and a Her2₁₈ gene were located upstream of EGFP were employed. In greater detail, HeLa cells were plated at a density of 2×10⁵ cells/well into 6-well plates containing 2 ml of DMEM supplemented with 10% FBS per well and cultured at 37° C. for 24 hrs in a 5% CO₂ atmosphere. When the cells were stabilized, each well was washed with 1 ml of PBS. Then, 800 μl of TOM (Transfection optimized medium, WelGENE Inc., Korea) was added to each well. After having reacted at room temperature for 20 min with 5 μl of Lipofectamine 2000 (Invitrogen, USA) in 200 μl of TOM medium in a tube, 500 ng of pEGFP-N1 or pEGFP-N1-G₁₈ alone was added to each well. Incubation was conducted at 37° C. for 6 hrs in a 5% CO₂ atmosphere, after which the medium was exchanged with 2 ml of DMEM supplemented with 10% FBS and the cells were further incubated for 24 hrs. Each well was washed with 1 ml of PBS. Then, 800 μl of TOM (Transfection optimized medium, WelGENE Inc., Korea) was added to each well. The cells were incubated at 37° C. for 2 hrs with the variants (10 μM) in a 5% CO₂ atmosphere. After removal of the medium, cells were obtained by treatment with trypsin-EDTA and washed with PBS. EGFP signals were detected using FACS Calibur (Fluorescent Activated Cell Sorter).

In addition, total RNAs and proteins were isolated from each sample and subjected to RT-PCR and Western blotting, respectively, to examine the downregulation of EGFP by 3D8 VL wild-type and the variants (4MG3, 4MG5) at protein and mRNA levels.

As for the variant 4MH2, it was analyzed by RT-PCR and Western-blotting as follows. In greater detail, HeLa cells were plated at a density of 2×10⁵ cells/well into 6-well plates containing 2 ml of DMEM supplemented with 10% FBS per well and cultured at 37° C. for 24 hrs in a 5% CO₂ atmosphere. After the cells were stabilized, each well was washed with 1 ml of PBS. Then, 800 μl of TOM (Transfection optimized medium, WelGENE Inc., Korea) was added to each well. After having reacted at room temperature for 20 min with 5 μl of Lipofectamine 2000 (Invitrogen, USA) in 200 μl of TOM medium in a tube, Her2 alone (500 nM) or Her2 on combination of siRNA (500 nM) was added to each well. Incubation was conducted at 37° C. for 6 hrs in a 5% CO₂ atmosphere, after which the medium was exchanged with 2 ml of DMEM supplemented with 10% FBS and the cells were incubated for 24 hrs. Each well was washed with 1 ml of PBS. Then, 800 μl of TOM (Transfection optimized medium, WelGENE Inc., Korea) was added to each well. The cells were incubated at 37° C. for 2 hrs with 3D8 VL WT and 4MH2 (10 μM) in a 5% CO₂ atmosphere. After removal of the medium, cells were obtained by treatment with trypsin-EDTA and washed with PBS. EGFP signals were detected using FACS Calibur (Fluorescent Activated Cell Sorter). Total RNA and proteins of interest were isolated from each sample and subjected to RT-PCR and Western blotting, respectively.

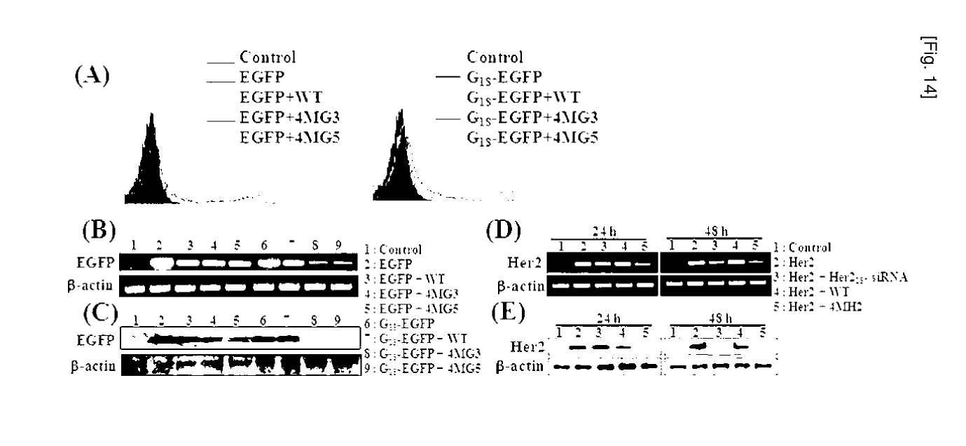

FIG. 14 shows target gene silencing activity of cell-penetrating 3D8 VL variants in HeLa cells expressing exogenous targeted genes. HeLa cells were untransfected (‘control’) or transfected with plasmids encoding EGFP or G₁₈-EGFP, and 12 h later either untreated or treated at 37° C. for 2 h with 3D8 VL WT (10 μM) and G₁₈-selective 4MG3 (10 μM) and 4MG5 (10 μM), and further incubated for 12 h before EGFP expression analyses by flow cytometry (A), RT-PCR (B, D), and Western blotting (C, E).

As shown in FIG. 14A, EGFP signal intensity did not significantly differ from 3D8 VL wild-type to 4MG3 and 4MG5 whereas transfection with the vector in which G₁₈ is located upstream of EGFP remarkably decreased EGFP signal intensity from the cells expressing 4MG3 or 4MG5 compared to the cells expressing 3D8 VL wild-type. Hence, upon cytosolic expression, the variants 4MG3 and 4MG5 can hydrolyze G₁₈-EGFP mRNA having the target base sequence thereof to downregulate EGFP expression.

Also, FIGS. 14B to 14E show the downregulation of GFP by intracellularly expressed variants (4MG3, 4MG5, 4MH2) at protein and mRNA levels. Hence, the variants (4MG3, 4MG5, 4MH2) are found to have base sequence specificity and nucleic acid-hydrolyzing activity.

### Example 11

### Cytotoxicity of the Variants (Proteins)

Cytotoxicity of the variants (proteins) were measured. In this regard, cells treated for a certain time with the variants (proteins) were measured for viability by MTT assay. Human breast carcinoma cells (SK-BR-3, MDA-MB-231) or human cervical carcinoma cells (HeLa) were plated at a density of 2×10⁴/well into 96-well plates containing 200 μl of DMEM supplemented with 10% FBS per well and cultured at 37° C. for 24 hrs in a 5% CO₂ atmosphere. When the cells stabilized, each well was washed with 200 μl of PBS. 80 μl of TOM (Transfection optimized medium, WelGENE Inc., Korea) was added to each well. After being treated with each variant (10 μM), the cells were monitored for viability for 24, 48 and 72 hrs.

In order to examine types of the cell death caused by the variants, each sample which had undergone the same procedure as described above was stained with FITC-Annexin V and PI and measured by FACS Calibur (Fluorescent Activated Cell Sorter).

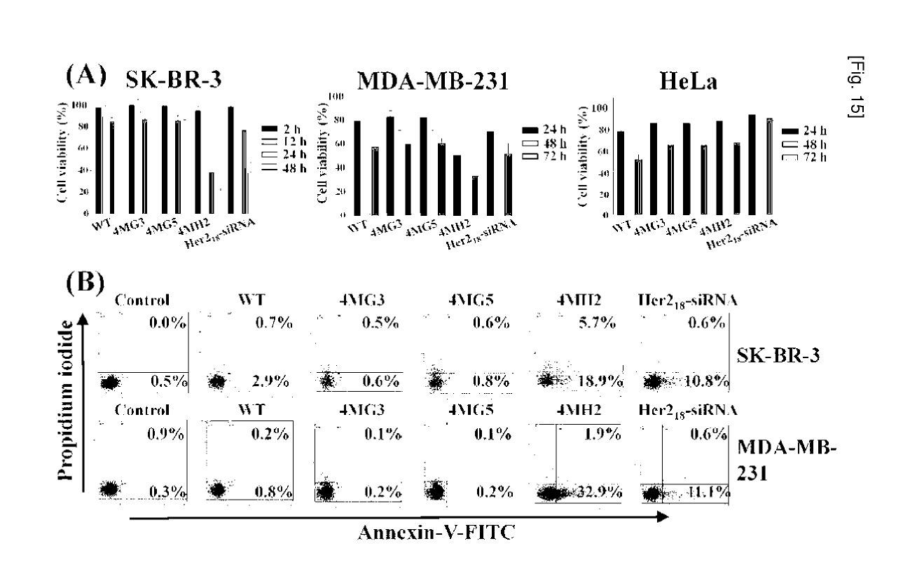

With reference to FIG. 15, human breast carcinoma cells (SK-BR-3, MDA-MB-231) or human cervical carcinoma cells (HeLa) treated with the variants were analyzed for viability by MTT assay (A) and FACS (B).

As shown in FIG. 15A, each antibody shows a low level of cytotoxicity. Particularly, the variant 4MH2, which can hydrolyze the Her2 base sequence with specificity therefor, was observed to exert potent cytotoxicity on Her2-expressing SK-BR-3 and MDA-MB-231, which is coincident with the previous report that Her2-overexpessing cells are decreased in cell viability as Her2 expression decreases. Thus, the downregulation of Her2 expression by 4MH2, in our opinion, decreased the cell viability.

As seen in FIG. 15B, each antibody shows toxicity to some degree, with coincidence with the results of FIG. 15A. 4MH2 and Her2 siRNA, both having nucleic acid-hydrolyzing activity with specificity for Her2 base sequence, were observed to be toxic to the Her2-overexpressing SK-BR-3 and MDA-MB-231 cells. At this time, the cells underwent apoptosis (Annexin V positive).

### Example 12

### Excellent Downregulation of Her2 Expression by the Variants (Proteins) with Her2 Base Sequence-Specific, Nucleic Acid-Hydrolyzing Activity

SK-BR-3, which overexpresses Her2, was employed for evaluating the downregulation of Her2 expression by the variants having Her2-specific, nucleic acid-hydrolyzing activity. In detail, SK-BR-3 cells were plated at a density of 2×10⁵ cells/well into 6-well plates containing 2 ml of DMEM supplemented with 10% FBS per well and cultured at 37° C. for 24 hrs in a 5% CO₂ atmosphere. When the cells were stabilized, each well was washed with 1 ml of PBS. Then, 800 μl of TOM (Transfection optimized medium, WelGENE Inc., Korea) was added to each well. The cells were incubated with each variant (10 μM) for 2, 12, 24 or 48 hrs. After removal of the medium, cells were obtained by treatment with trypsin-EDTA and washed with PBS. The expression levels of Her2 proteins on the cell surface were detected with FACS Calibur (Fluorescent Activated Cell Sorter).

Total RNA or proteins were isolated from each sample and subjected to RT-PCR and Western blotting, respectively, by which the 4MH2 antibody was again observed to hydrolyze nucleic acids, with the retention of base sequence specificity.

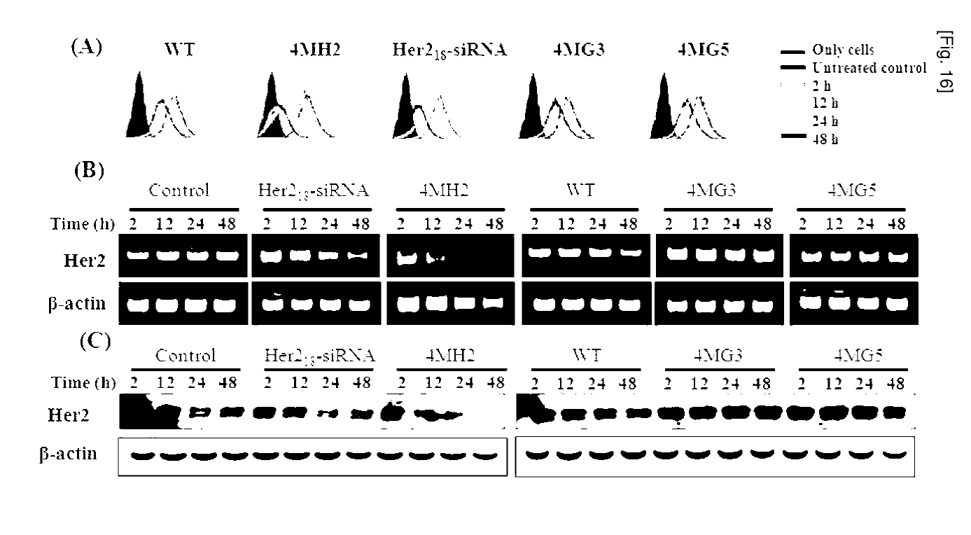

In FIG. 16, Her2 expression levels in the presence of 4MH2, cell-penetrating Her2₁₈-selective variant, having Her2 sequence-specific, nucleic acid-hydrolyzing activity in Her2-overexpressing SK-BR-3 cells were analyzed by FACS (A), RT-PCR (B), and Western blotting (C).

As seen in FIG. 16A, 4MH2 selectively decreased the expression level of the cell surface protein Her2. Starting from 2 hrs post-transfection, the time needed for the sufficient internalization of the variants and the downregulation required 48 hrs to reach a peak. Compared to the positive control Her2 siRNA, 4MH2 was observed to exert higher downregulation from an earlier time, indicating the superiority of 4MH2 to Her2 siRNA in terms of activity and time.

Also, FIGS. 16B and 16C show that Her2 expression on cell surfaces was reduced selectively by 4MH2, as in FIG. 16A. Faster and stronger downregulation was observed in 4MH2 than in Her2 siRNA.

### INDUSTRIAL APPLICABILITY

As described hitherto, the nucleic acid-hydrolyzing antibodies in accordance with the present invention can be prepared by modifying a particular site of a cell-penetrating, nucleic acid-hydrolyzing antibody which lacks substrate specificity to impart sequence specificity thereto without alteration in nucleic acid-hydrolyzing ability. The engineered nucleic acid-hydrolyzing antibodies, when penetrating into cells by themselves or expressed within cells, bind specifically to single- or double-stranded nucleic acid targets and hydrolyze them, thus downregulating the expression of target genes. Therefore, the nucleic acid-hydrolyzing antibodies according to the present invention can be an alternative to or a substitute for conventional gene silencing techniques such as siRNA. Particularly, the nucleic acid-hydrolyzing antibodies of the present invention can downregulate the expression of target proteins or the proliferation of target genomes at RNA or DNA levels, but not at protein levels, by binding specifically to and hydrolyzing RNA or DNA, so that they are useful as therapeutics for cancers and viral diseases. Accordingly, the nucleic acid-hydrolyzing antibodies of the present invention may be developed into novel anticancer drugs or anti-viral drugs.

### SEQUENCE LISTING FREE TEXT

| | |
|---|---|
| <160> 41 | |
| <170> KopatentIn 1.71 | |
| <210> 1 | |
| <211> 113 | |
| <212> PRT | |
| <213> Artificial Sequence | |
| <220> | |
| <223> amino acid sequence of 3D8 VL WT | |
| <400> 1 | |
| Asp Leu Val Met Ser Gln Ser Pro Ser Ser Leu Ala Val Ser Ala Gly | |
| 1 5 10 15 | |
| Glu Lys Val Thr Met Ser Cys Lys Ser Ser Gln Ser Leu Phe Asn Ser | |
| 20 25 30 | |
| Arg Thr Arg Lys Asn Tyr Leu Ala Trp Tyr Gln Gln Lys Pro Gly Gln | |
| 35 40 45 | |
| Ser Pro Lys Leu Leu Ile Tyr Trp Ala Ser Thr Arg Glu Ser Gly Val | |
| 50 55 60 | |
| Pro Asp Arg Phe Thr Gly Ser Gly Ser Gly Thr Asp Phe Thr Leu Thr | |
| 65 70 75 80 | |
| Ile Ser Ser Val Gln Ala Glu Asp Leu Ala Val Tyr Tyr Cys Lys Gln | |
| 85 90 95 | |
| Ser Tyr Tyr His Met Tyr Thr Phe Gly Ser Gly Thr Lys Leu Glu Ile | |
| 100 105 110 | |
| Lys | |
| <210> 2 | |
| <211> 113 | |
| <212> PRT | |
| <213> Artificial Sequence | |
| <220> | |
| <223> amino acid sequence of 3D8 VL 4M | |
| <400> 2 | |
| Asp Leu Val Met Ser Gln Ser Pro Ser Ser Leu Ala Val Ser Ala Gly | |
| 1 5 10 15 | |
| Glu Lys Val Thr Met Ser Cys Lys Ser Ser Gln Ser Leu Phe Asn Ser | |
| 20 25 30 | |
| Arg Thr Arg Lys Asn Tyr Leu Ala Trp Tyr Gln Gln Lys Pro Gly Arg | |
| 35 40 45 | |
| Ser Pro Lys Leu Leu Ile His Arg Ala Ser Thr Arg Glu Ser Gly Val | |
| 50 55 60 | |
| Pro Asp Arg Phe Thr Gly Ser Gly Ser Gly Thr Asp Phe Thr Leu Thr | |
| 65 70 75 80 | |
| Ile Ser Ser Val Gln Ala Glu Asp Leu Ala Val Tyr Tyr Cys Lys Gln | |
| 85 90 95 | |
| Ser Tyr Tyr Ala Met Tyr Thr Phe Gly Ser Gly Thr Lys Leu Glu Ile | |
| 100 105 110 | |
| Lys | |
| <210> 3 | |
| <211> 339 | |
| <212> DNA | |
| <213> Artificial Sequence | |
| <220> | |
| <223> nucleotide sequence of 3D8 VL WT | |
| <400> 3 | |
| gatcttgtga tgtcacagtc tccatcctcc ctggctgtgt cagcaggaga gaaggtcact | 60 |
| atgagctgca aatccagtca gagtctgttc aacagtagaa cccgaaagaa ctacttggct | 120 |
| tggtaccagc agaaaccagg gcagtctcct aaactgctga tctactgggc atccactagg | 180 |
| gaatctgggg tccctgatcg cttcacaggc agtggatctg ggacagattt cactctcacc | 240 |
| atcagcagtg tgcaggctga agacctggca gtttattact gcaagcaatc ttattatcac | 300 |
| atgtatacgt tcggatcggg gaccaagctg gaaataaaa | 339 |
| <210> 4 | |
| <211> 339 | |
| <212> DNA | |
| <213> Artificial Sequence | |
| <220> | |
| <223> nucleotide sequence of 3D8 VL 4M | |
| <400> 4 | |
| gatcttgtga tgtcacagtc tccatcctcc ctggctgtgt cagcaggaga gaaggtcact | 60 |
| atgagctgca aatccagtca gagtctgttc aacagtagaa cccgaaagaa ctacttggct | 120 |
| tggtaccagc agaaaccagg gcggtctcct aaactgctga tccaccgggc atccactagg | 180 |
| gaatctgggg tccctgatcg cttcacaggc agtggatctg ggacagattt cactctcacc | 240 |
| atcagcagtg tgcaggctga agacctggca gtttattact gcaagcaatc ttattatgcc | 300 |
| atgtatacgt tcggatcggg gaccaagctg gaaataaaa | 339 |
| <210> 5 | |
| <211> 24 | |
| <212> DNA | |
| <213> Artificial Sequence | |
| <220> | |
| <223> nucleotide sequence of primer 1F | |
| <400> 5 | |
| caggctagtg gtggtggtgg ttct | 24 |
| <210> 6 | |
| <211> 60 | |
| <212> DNA | |
| <213> Artificial Sequence | |
| <220> | |
| <223> nucleotide sequence of primer 2R | |
| <400> 6 | |
| cagcagttta ggagaccgcc ctggvnnvnn vnnvnnvnna gccaagtagt tctttcgggt | 60 |
| 60 | |
| <210> 7 | |
| <211> 51 | |
| <212> DNA | |
| <213> Artificial Sequence | |
| <220> | |
| <223> nucleotide sequence of primer 3R | |
| <400> 7 | |
| agattcccta gtggatgccc ggtgvnnvnn vnnvnnvnna gaccgccctg g | 51 |
| <210> 8 | |
| <211> 24 | |
| <212> DNA | |
| <213> Artificial Sequence | |
| <220> | |
| <223> nucleotide sequence of primer 4F | |
| <400> 8 | |
| caccgggcat ccactaggga atct | 24 |
| <210> 9 | |
| <211> 24 | |
| <212> DNA | |
| <213> Artificial Sequence | |
| <220> | |
| <223> nucleotide sequence of primer 5R | |
| <400> 9 | |
| caggtcttca gcctgcacac tgct | 24 |
| <210> 10 | |
| <211> 60 | |
| <212> DNA | |
| <213> Artificial Sequence | |
| <220> | |
| <223> nucleotide sequence of primer 6F | |
| <400> 10 | |
| agcagtgtgc aggctgaaga cctgnnbnnb nnbnnbnnba agcaatctta ttatgccatg | 60 |
| 60 | |
| <210> 11 | |
| <211> 30 | |
| <212> DNA | |
| <213> Artificial Sequence | |
| <220> | |
| <223> nucleotide sequence of primer 7R | |
| <400> 11 | |
| gatctcgcgc tattacaagt cctcttcaga | 30 |
| <210> 12 | |
| <211> 18 | |
| <212> DNA | |
| <213> Artificial Sequence | |
| <220> | |
| <223> nucleotide sequence of 5′-biotinylated substrate(G(18)) | |
| <400> 12 | |
| gggggggggg gggggggg | 18 |
| <210> 13 | |
| <211> 18 | |
| <212> DNA | |
| <213> Artificial Sequence | |
| <220> | |
| <223> nucleotide sequence of 5′-biotinylated substrate(Her2(18)) | |
| <400> 13 | |
| aattccagtg gccatcaa | 18 |
| <210> 14 | |
| <211> 113 | |
| <212> PRT | |
| <213> Artificial Sequence | |
| <220> | |
| <223> amino acid sequence of 3D8 VL mutant(4MG1) | |
| <400> 14 | |
| Asp Leu Val Met Ser Gln Ser Pro Ser Ser Leu Ala Val Ser Ala Gly | |
| 1 5 10 15 | |
| Glu Lys Val Thr Met Ser Cys Lys Ser Ser Gln Ser Leu Phe Asn Ser | |
| 20 25 30 | |
| Arg Thr Arg Lys Asn Tyr Leu Ala Trp Asn Gln Arg Lys Pro Gly Arg | |
| 35 40 45 | |
| Ser Arg Lys Ser Leu Ile His Arg Ala Ser Thr Arg Glu Pro Gly Val | |
| 50 55 60 | |
| Pro Asp Arg Phe Thr Gly Ser Gly Ser Gly Thr Asp Phe Thr Leu Thr | |
| 65 70 75 80 | |
| Ile Ser Ser Val Glu Pro Glu Glu Leu Ala Gly Tyr Tyr Cys Lys Gln | |
| 85 90 95 | |
| Cys Tyr Tyr Ala Met Tyr Thr Phe Gly Ser Gly Thr Lys Leu Glu Ile | |
| 100 105 110 | |
| Lys | |
| <210> 15 | |
| <211> 113 | |
| <212> PRT | |
| <213> Artificial Sequence | |
| <220> | |
| <223> amino acid sequence of 3D8 VL mutant(4MG2) | |
| <400> 15 | |
| Asp Leu Val Met Ser Gln Ser Pro Ser Ser Leu Ala Val Ser Ala Gly | |
| 1 5 10 15 | |
| Glu Lys Val Thr Met Ser Cys Lys Ser Ser Gln Ser Leu Phe Asn Ser | |
| 20 25 30 | |
| Arg Thr Arg Lys Asn Tyr Leu Ala Trp Gln Gln Arg Lys Pro Gly Arg | |
| 35 40 45 | |
| Ser Arg Lys Arg Leu Ile His Arg Ala Ser Thr Arg Glu Ser Gly Val | |
| 50 55 60 | |
| Pro Asp Arg Phe Thr Gly Ser Gly Ser Gly Thr Asp Phe Thr Leu Thr | |
| 65 70 75 80 | |
| Ile Ser Ser Val Gln Ala Glu Glu Val Gly Arg Gly Gly Asp Lys Gln | |
| 85 90 95 | |
| Ser Tyr Tyr Ala Met Tyr Thr Phe Gly Ser Gly Thr Lys Leu Glu Ile | |
| 100 105 110 | |
| Lys | |
| <210> 16 | |
| <211> 113 | |
| <212> PRT | |
| <213> Artificial Sequence | |
| <220> | |
| <223> amino acid sequence of 3D8 VL mutant(4MG3) | |
| <400> 16 | |
| Asp Leu Val Met Ser Gln Ser Pro Ser Ser Leu Ala Val Ser Ala Gly | |
| 1 5 10 15 | |
| Glu Lys Val Thr Met Ser Cys Lys Ser Ser Gln Ser Leu Phe Asn Ser | |
| 20 25 30 | |
| Arg Ala Arg Lys Asn Tyr Leu Ala Trp Arg Gln Lys Lys Pro Gly Arg | |
| 35 40 45 | |
| Ser Arg Lys Gln Leu Ile His Arg Ala Ser Thr Arg Glu Ser Gly Val | |
| 50 55 60 | |
| Pro Asp Arg Phe Thr Gly Ser Gly Ser Gly Thr Asp Phe Thr Leu Thr | |
| 65 70 75 80 | |
| Ile Ser Ser Val Gln Ala Glu Glu Leu Arg Glu Glu Asn Arg Lys Glu | |
| 85 90 95 | |
| Ser Tyr Tyr Ala Met Tyr Thr Phe Gly Ser Gly Thr Lys Leu Glu Ile | |
| 100 105 110 | |
| Lys | |
| <210> 17 | |
| <211> 113 | |
| <212> PRT | |
| <213> Artificial Sequence | |
| <220> | |
| <223> amino acid sequence of 3D8 VL mutant(4MG4) | |
| <400> 17 | |
| Asp Leu Val Met Ser Gln Ser Pro Ser Ser Leu Ala Val Ser Ala Gly | |
| 1 5 10 15 | |
| Glu Lys Val Thr Met Ser Cys Lys Ser Ser Gln Ser Leu Phe Asn Ser | |
| 20 25 30 | |
| Arg Thr Arg Lys Asn Tyr Leu Ala Asn Asn Arg Arg Arg Pro Gly Arg | |
| 35 40 45 | |
| Ser Arg Asn Lys His Glu His Arg Ala Ser Thr Arg Glu Ser Gly Val | |
| 50 55 60 | |
| Pro Asp Arg Phe Thr Gly Ser Gly Ser Gly Thr Asp Phe Thr Leu Thr | |
| 65 70 75 80 | |
| Ile Ser Ser Val Gln Gly Glu Glu Leu Pro Glu Asp Pro His Lys Gln | |
| 85 90 95 | |
| Ser Tyr Tyr Ala Met Tyr Thr Phe Gly Ser Gly Thr Lys Leu Glu Ile | |
| 100 105 110 | |
| Lys | |
| <210> 18 | |
| <211> 113 | |
| <212> PRT | |
| <213> Artificial Sequence | |
| <220> | |
| <223> amino acid sequence of 3D8 VL mutant(4MG5) | |
| <400> 18 | |
| Asp Leu Val Met Ser Gln Ser Pro Ser Ser Leu Ala Val Ser Ala Gly | |
| 1 5 10 15 | |
| Glu Lys Val Thr Met Ser Cys Lys Ser Ser Gln Ser Leu Phe Asn Ser | |
| 20 25 30 | |
| Arg Thr Arg Lys Asn Tyr Leu Ala Lys Asn Gln Gly Gln Pro Gly Arg | |
| 35 40 45 | |
| Ser Arg Lys Asn Asn Arg His Arg Ala Ser Thr Arg Glu Ser Gly Val | |
| 50 55 60 | |
| Pro Asp Arg Phe Thr Gly Ser Gly Ser Gly Thr Asp Phe Thr Leu Thr | |
| 65 70 75 80 | |
| Ile Ser Ser Val Gln Ala Glu Asp Leu Gly Arg Tyr Asn Ser Asn Gln | |
| 85 90 95 | |
| Ser Tyr Tyr Ala Met Tyr Thr Phe Gly Ser Gly Thr Lys Leu Glu Ile | |
| 100 105 110 | |
| Lys | |
| <210> 19 | |
| <211> 113 | |
| <212> PRT | |
| <213> Artificial Sequence | |
| <220> | |
| <223> amino acid sequence of 3D8 VL mutant(4MG6) | |
| <400> 19 | |
| Asp Leu Val Met Ser Gln Ser Pro Ser Ser Leu Ala Val Ser Ala Gly | |
| 1 5 10 15 | |
| Glu Lys Val Thr Met Ser Cys Lys Ser Ser Gln Ser Leu Phe Asn Ser | |
| 20 25 30 | |
| Arg Thr Arg Lys Asn Tyr Leu Ala Ser Arg Lys Arg Gly Pro Gly Arg | |
| 35 40 45 | |
| Ser Gly Lys Asn His Arg His Arg Ala Ser Thr Arg Glu Ser Gly Val | |
| 50 55 60 | |
| Pro Asp Arg Phe Thr Gly Ser Gly Ser Gly Thr Asp Phe Thr Leu Thr | |
| 65 70 75 80 | |
| Ile Ser Ser Val Glu Gly Glu Asp Leu Gly Glu Tyr Trp Cys Lys Glu | |
| 85 90 95 | |
| Ser Tyr Tyr Ala Met Tyr Thr Phe Gly Ser Gly Thr Lys Leu Glu Ile | |
| 100 105 110 | |
| Lys | |
| <210> 20 | |
| <211> 113 | |
| <212> PRT | |
| <213> Artificial Sequence | |
| <220> | |
| <223> amino acid sequence of 3D8 VL mutant(4MH1) | |
| <400> 20 | |
| Asp Leu Val Met Ser Gln Ser Pro Ser Ser Leu Ala Val Ser Ala Gly | |
| 1 5 10 15 | |
| Glu Lys Val Thr Met Ser Cys Lys Ser Ser Gln Ser Leu Phe Asn Ser | |
| 20 25 30 | |
| Arg Thr Arg Lys Asn Tyr Leu Ala Ser Lys Glu Lys His Pro Gly Arg | |
| 35 40 45 | |
| Ser Asn Gly Ser Arg Gln His Arg Ala Ser Thr Arg Glu Ser Gly Val | |
| 50 55 60 | |
| Pro Asp Arg Phe Thr Gly Ser Gly Ser Gly Thr Asp Phe Thr Leu Thr | |
| 65 70 75 80 | |
| Ile Ser Ser Val Gln Ala Glu Glu Leu Ala Tyr Tyr Asn Cys Lys Gln | |
| 85 90 95 | |
| Ser Tyr Tyr Ala Met Tyr Thr Phe Gly Ser Gly Thr Lys Leu Glu Ile | |
| 100 105 110 | |
| Lys | |
| <210> 21 | |
| <211> 113 | |
| <212> PRT | |
| <213> Artificial Sequence | |
| <220> | |
| <223> amino acid sequence of 3D8 VL mutant(4MH2) | |
| <400> 21 | |
| Asp Leu Val Met Ser Gln Ser Pro Ser Ser Leu Ala Val Ser Ala Gly | |
| 1 5 10 15 | |
| Glu Lys Val Thr Met Ser Cys Lys Ser Ser Gln Ser Leu Phe Asn Ser | |
| 20 25 30 | |
| Arg Thr Arg Lys Asn Tyr Leu Ala Trp Asn Gln Cys Lys Pro Gly Arg | |
| 35 40 45 | |
| Ser Glu Lys Asn Leu Ile His Arg Ala Ser Thr Arg Glu Ser Gly Val | |
| 50 55 60 | |
| Pro Asp Arg Phe Thr Gly Ser Gly Ser Gly Thr Asp Phe Thr Leu Thr | |
| 65 70 75 80 | |
| Ile Ser Ser Val Gln Ala Glu Asp Leu Asp Ile Gln Gln Ala Lys Gln | |
| 85 90 95 | |
| Cys Tyr Tyr Ala Met Tyr Thr Phe Gly Ser Gly Thr Lys Leu Glu Ile | |
| 100 105 110 | |
| Lys | |
| <210> 22 | |
| <211> 113 | |
| <212> PRT | |
| <213> Artificial Sequence | |
| <220> | |
| <223> amino acid sequence of 3D8 VL mutant(4MH3) | |
| <400> 22 | |
| Asp Leu Val Met Ser Gln Ser Pro Ser Ser Leu Ala Val Ser Ala Gly | |
| 1 5 10 15 | |
| Glu Lys Val Thr Met Ser Cys Lys Ser Ser Gln Ser Leu Phe Asn Ser | |
| 20 25 30 | |
| Arg Thr Arg Lys Asn Tyr Leu Ala Ser Glu Arg Lys Arg Pro Gly Arg | |
| 35 40 45 | |
| Ser Glu Asn Asn Arg Arg His Arg Ala Ser Thr Arg Glu Ser Gly Val | |
| 50 55 60 | |
| Pro Asp Arg Phe Thr Gly Ser Gly Ser Gly Thr Asp Phe Thr Leu Thr | |
| 65 70 75 80 | |
| Ile Ser Ser Val Gln Ala Gln Asp Leu Gly Asp Gln Gln Gly Lys Glu | |
| 85 90 95 | |
| Cys Tyr Tyr Ala Met Tyr Thr Phe Gly Ser Gly Thr Lys Leu Glu Ile | |
| 100 105 110 | |
| Lys | |
| <210> 23 | |
| <211> 113 | |
| <212> PRT | |
| <213> Artificial Sequence | |
| <220> | |
| <223> amino acid sequence of 3D8 VL mutant(4MH4) | |
| <400> 23 | |
| Asp Leu Val Met Ser Gln Ser Pro Ser Ser Leu Ala Val Ser Ala Gly | |
| 1 5 10 15 | |
| Glu Lys Val Thr Met Ser Cys Lys Ser Ser Gln Ser Leu Phe Asn Ser | |
| 20 25 30 | |
| Arg Thr Arg Lys Asn Tyr Leu Ala Trp Tyr Gln His Lys Pro Gly Arg | |
| 35 40 45 | |
| Ser Gly Lys Ser Leu Ile His Arg Ala Ser Thr Arg Glu Ser Gly Val | |
| 50 55 60 | |
| Pro Asp Arg Phe Thr Gly Ser Gly Ser Gly Thr Asp Phe Thr Leu Thr | |
| 65 70 75 80 | |
| Ile Ser Ser Val Gln Ala Glu Asp Leu Gly Asn Tyr Gly Cys Lys Glu | |
| 85 90 95 | |
| Cys Tyr Tyr Ala Met Tyr Thr Phe Gly Ser Gly Thr Lys Leu Glu Ile | |
| 100 105 110 | |
| Lys | |
| <210> 24 | |
| <211> 113 | |
| <212> PRT | |
| <213> Artificial Sequence | |
| <220> | |
| <223> amino acid sequence of 3D8 VL mutant(4MH5) | |
| <400> 24 | |
| Asp Leu Val Met Ser Gln Ser Pro Ser Ser Leu Ala Val Ser Ala Gly | |
| 1 5 10 15 | |
| Glu Lys Val Thr Met Ser Cys Lys Ser Ser Gln Ser Leu Phe Asn Ser | |
| 20 25 30 | |
| Arg Thr Arg Lys Asn Tyr Leu Ala Trp Tyr Gln Gln Lys Pro Gly Arg | |
| 35 40 45 | |
| Ser Ser Lys Gly Leu Ile His Arg Ala Ser Thr Arg Glu Ser Gly Val | |
| 50 55 60 | |
| Pro Asp Arg Phe Thr Gly Ser Gly Ser Gly Thr Asp Phe Thr Leu Thr | |
| 65 70 75 80 | |
| Ile Ser Ser Val Gln Ala Glu Glu Leu Arg Gly Lys Arg Gly Lys Gln | |
| 85 90 95 | |
| Cys Tyr Tyr Ala Met Tyr Thr Phe Gly Ser Gly Thr Lys Leu Glu Ile | |
| 100 105 110 | |
| Lys | |
| <210> 25 | |
| <211> 339 | |
| <212> DNA | |
| <213> Artificial Sequence | |
| <220> | |
| <223> nucleotide sequence of 3D8 VL mutant(4MG1) | |
| <400> 25 | |
| gatcttgtga tgtcacagtc tccatcctcc ctggctgtgt cagcaggaga gaaggtcact | 60 |
| atgagctgca aatccagtca gagtctgttc aacagtagaa cccgaaagaa ctacttggct | 120 |
| tggaaccagc gcaaaccagg gcggtctcgc aaaagcctga tccaccgggc atccaccagg | 180 |
| gaacctgggg tccctgatcg cttcacaggc agtggatctg ggacagattt cactctcacc | 240 |
| atcagcagtg tggagcctga agagctggca gggtattact gcaagcaatg ttattatgcc | 300 |
| atgtatacgt tcggatcggg gaccaagctg gaaataaaa | 339 |
| <210> 26 | |
| <211> 339 | |
| <212> DNA | |
| <213> Artificial Sequence | |
| <220> | |
| <223> nucleotide sequence of 3D8 VL mutant(4MG2) | |
| <400> 26 | |
| gatcttgtga tgtcacagtc tccatcctcc ctggctgtgt cagcaggaga gaaggtcact | 60 |
| atgagctgca aatccagtca gagtctgttc aacagtagaa cccgaaagaa ctacttggct | 120 |
| tggcagcagc gtaaaccagg gcggtctcgc aaacgcctga tccaccgggc atccactagg | 180 |
| gaatctgggg tccctgatcg cttcacaggc agtggatctg ggacagattt cactctcacc | 240 |
| atcagcagtg tgcaggctga agaggtgggt cggggtgggg acaagcaatc ttattatgcc | 300 |
| atgtatacgt tcggatcggg gaccaagctg gaaataaaa | 339 |
| <210> 27 | |
| <211> 339 | |
| <212> DNA | |
| <213> Artificial Sequence | |
| <220> | |
| <223> nucleotide sequence of 3D8 VL mutant(4MG3) | |
| <400> 27 | |
| gatcttgtga tgtcacagtc tccatcctcc ctggctgtgt cagcaggaga gaaggtcact | 60 |
| atgagctgca aatccagtca gagtctgttc aacagtagag cccgaaagaa ctacttggct | 120 |
| tggaggcaga agaaaccagg gcggtctcgc aaacagctga tccaccgggc atccactagg | 180 |
| gaatctgggg tccctgatcg cttcacaggc agtggatctg ggacagattt cactctcacc | 240 |
| atcagcagtg tgcaggctga agagctgagg gaagaaaacc ggaaggaatc ttattatgcc | 300 |
| atgtatacgt tcggatcggg gaccaagctg gaaataaaa | 339 |
| <210> 28 | |
| <211> 339 | |
| <212> DNA | |
| <213> Artificial Sequence | |
| <220> | |
| <223> nucleotide sequence of 3D8 VL mutant(4MG4) | |
| <400> 28 | |
| gatcttgtga tgtcacagtc tccatcctcc ctggctgtgt cagcaggaga gaaggtcact | 60 |
| atgagctgca aatccagtca gagtctgttc aacagtagaa cccgaaagaa ctacttggct | 120 |
| aataacaggc gtaggccagg gcggtctcgg aataaacatg aacaccgggc atccactagg | 180 |
| gaatctgggg tccctgatcg cttcacaggc agtggatctg ggacagattt cactctcacc | 240 |
| atcagcagtg tgcagggtga agagctgccg gaggatcctc acaagcaatc ttattatgcc | 300 |
| atgtatacgt tcggatcggg gaccaagctg gaaataaaa | 339 |
| <210> 29 | |
| <211> 339 | |
| <212> DNA | |
| <213> Artificial Sequence | |
| <220> | |
| <223> nucleotide sequence of 3D8 VL mutant(4MG5) | |
| <400> 29 | |
| gatcttgtga tgtcacagtc tccatcctcc ctggctgtgt cagcaggaga gaaggtcact | 60 |
| atgagctgca aatccagtca gagtctgttc aacagtagaa cccgaaagaa ctacttggct | 120 |
| aaaaatcaag gacaaccagg gcggtctaga aaaaacaaca ggcaccgggc atccactagg | 180 |
| gaatctgggg tccctgatcg cttcacaggc agtggatctg ggacagattt cactctcacc | 240 |
| atcagcagtg tgcaggctga agacctggga cgttataatt ccaaccaatc ttattatgcc | 300 |
| atgtatacgt tcggatcggg gaccaagctg gaaataaaa | 339 |
| <210> 30 | |
| <211> 339 | |
| <212> DNA | |
| <213> Artificial Sequence | |
| <220> | |
| <223> nucleotide sequence of 3D8 VL mutant(4MG6) | |
| <400> 30 | |
| gatcttgtga tgtcacagtc tccatcctcc ctggctgtgt cagcaggaga gaaggtcact | 60 |
| atgagctgca aatccagtca gagtctgttc aacagtagaa cccgaaagaa ctacttggct | 120 |
| agtagaaagc gaggaccagg gcggtctggt aagaaccaca gacaccgggc atccactagg | 180 |
| gaatctgggg tccctgatcg cttcacaggc agtggatctg ggacagattt cactctcacc | 240 |
| atcagcagtg tggagggtga agacctggga gagtattggt gcaaggaatc ttattatgcc | 300 |
| atgtatacgt tcggatcggg gaccaagctg gaaataaaa | 339 |
| <210> 31 | |
| <211> 339 | |
| <212> DNA | |
| <213> Artificial Sequence | |
| <220> | |
| <223> nucleotide sequence of 3D8 VL mutant(4MH1) | |
| <400> 31 | |
| gatcttgtga tgtcacagtc tccatcctcc ctggctgtgt cagcaggaga gaaggtcact | 60 |
| atgagctgca aatccagtca gagtctgttc aacagtagaa cccgaaagaa ctacttggct | 120 |
| agtaaggaaa aacacccagg gcggtctaac ggcagccgac agcaccgggc atccactagg | 180 |
| gaatctgggg tccctgatcg cttcacaggc agtggatctg ggacagattt cactctcacc | 240 |
| atcagcagtg tgcaggctga agagctggca tattataact gcaagcaatc ttattatgcc | 300 |
| atgtatacgt tcggatcggg gaccaagctg gaaataaaa | 339 |
| <210> 32 | |
| <211> 339 | |
| <212> DNA | |
| <213> Artificial Sequence | |
| <220> | |
| <223> nucleotide sequence of 3D8 VL mutant(4MH2) | |
| <400> 32 | |
| gatcttgtga tgtcacagtc tccatcctcc ctggctgtgt cagcaggaga gaaggtcact | 60 |
| atgagctgca aatccagtca gagtctgttc aacagtagaa cccgaaagaa ctacttggct | 120 |
| tggaaccagt gcaaaccagg gcggtctgag aaaaatctga tccaccgggc atccactagg | 180 |
| gaatctgggg tccctgatcg cttcacaggc agtggatctg ggacagattt cactctcacc | 240 |
| atcagcagtg tgcaggctga agacctggat attcagcaag cgaagcaatg ttattatgcc | 300 |
| atgtatacgt tcggatcggg gaccaagctg gaaataaaa | 339 |
| <210> 33 | |
| <211> 339 | |
| <212> DNA | |
| <213> Artificial Sequence | |
| <220> | |
| <223> nucleotide sequence of 3D8 VL mutant(4MH3) | |
| <400> 33 | |
| gatcttgtga tgtcacagtc tccatcctcc ctggctgtgt cagcaggaga gaaggtcact | 60 |
| atgagctgca aatccagtca gagtctgttc aacagtagaa cccgaaagaa ctacttggct | 120 |
| agtgagcgaa agcgaccagg gcggtctgag aataacaggc ggcaccgggc atccactagg | 180 |
| gaatctgggg tccctgatcg cttcacaggc agtggatctg ggacagattt cactctcacc | 240 |
| atcagcagtg tgcaggctca agacctgggt gatcagcaag ggaaggaatg ttattatgcc | 300 |
| atgtatacgt tcggatcggg gaccaagctg gaaataaaa | 339 |
| <210> 34 | |
| <211> 339 | |
| <212> DNA | |
| <213> Artificial Sequence | |
| <220> | |
| <223> nucleotide sequence of 3D8 VL mutant(4MH4) | |
| <400> 34 | |
| gatcttgtga tgtcacagtc tccatcctcc ctggctgtgt cagcaggaga gaaggtcact | 60 |
| atgagctgca aatccagtca gagtctgttc aacagtagaa cccgaaagaa ctacttggct | 120 |
| tggtaccagc ataaaccagg gcggtctggc aaaagtctga tccaccgggc atccactagg | 180 |
| gaatctgggg tccctgatcg cttcacaggc agtggatctg ggacagattt cactctcacc | 240 |
| atcagcagtg tgcaggctga agacctggga aactatggtt gcaaggaatg ttattatgcc | 300 |
| atgtatacgt tcggatcggg gaccaagctg gaaataaaa | 339 |
| <210> 35 | |
| <211> 339 | |
| <212> DNA | |
| <213> Artificial Sequence | |
| <220> | |
| <223> nucleotide sequence of 3D8 VL mutant(4MH5) | |
| <400> 35 | |
| gatcttgtga tgtcacagtc tccatcctcc ctggctgtgt cagcaggaga gaaggtcact | 60 |
| atgagctgca aatccagtca gagtctgttc aacagtagaa cccgaaagaa ctacttggct | 120 |
| tggtaccagc agaaaccagg gcggtctagc aaagggctga tccaccgggc atccactagg | 180 |
| gaatctgggg tccctgatcg cttcacaggc agtggatctg ggacagattt cactctcacc | 240 |
| atcagcagtg tgcaggctga agagctgagg gggaagcggg gcaagcaatg ttattatgcc | 300 |
| atgtatacgt tcggatcggg gaccaagctg gaaataaaa | 339 |
| <210> 36 | |
| <211> 18 | |
| <212> DNA | |
| <213> Artificial Sequence | |
| <220> | |
| <223> nucleotide sequence of FRET substrate(A18) which was labeled with | |
| 6-FAM at 5′-terminus and BHQ-1 at 3′-terminus | |
| <400> 36 | |
| aaaaaaaaaa aaaaaaaa | 18 |
| <210> 37 | |
| <211> 18 | |
| <212> DNA | |
| <213> Artificial Sequence | |
| <220> | |
| <223> nucleotide sequence of FRET substrate(T18) which was labeled with | |
| 6-FAM at 5′-terminus and BHQ-1 at 3′-terminus | |
| <400> 37 | |
| tttttttttt tttttttt | 18 |
| <210> 38 | |
| <211> 18 | |
| <212> DNA | |
| <213> Artificial Sequence | |
| <220> | |
| <223> nucleotide sequence of FRET substrate(C18) which was labeled with | |
| 6-FAM at 5′-terminus and BHQ-1 at 3′-terminus | |
| <400> 38 | |
| cccccccccc cccccccc | 18 |
| <210> 39 | |
| <211> 18 | |
| <212> DNA | |
| <213> Artificial Sequence | |
| <220> | |
| <223> nucleotide sequence of FRET substrate((G4T)3G3) which was labeled | |
| with 6-FAM at 5′-terminus and BHQ-1 at 3′-terminus | |
| <400> 39 | |
| ggggtggggt ggggtggg | 18 |
| <210> 40 | |
| <211> 18 | |
| <212> DNA | |
| <213> Artificial Sequence | |
| <220> | |
| <223> nucleotide sequence of FRET substrate(Her2(18)) which was labeled | |
| with 6-FAM at 5′-terminus and BHQ-1 at 3′-terminus | |
| <400> 40 | |
| aattccagtg gccatcaa | 18 |
| <210> 41 | |
| <211> 18 | |
| <212> DNA | |
| <213> Artificial Sequence | |
| <220> | |
| <223> nucleotide sequence of FRET substrate(N18) which was labeled with | |
| 6-FAM at 5′-terminus and BHQ-1 at 3′-terminus | |
| <400> 41 | |
| actgactgac tgactgac | 18 |

1. A nucleic acid-hydrolyzing antibody, capable of penetrating into cells and specifically binding to a single- or double-stranded nucleic acid target of a particular base sequence, and hydrolyzing the targeted nucleic acid.

2. The nucleic acid-hydrolyzing antibody according to claim 1, wherein the target is G₁₈ or Her2₁₈.

3. The nucleic acid-hydrolyzing antibody according to claim 2, wherein the G₁₈ has a base sequence of SEQ ID NO: 12.

4. The nucleic acid-hydrolyzing antibody according to claim 2, wherein the Her2₁₈ has a base sequence of SEQ ID NO: 13.

5. The nucleic acid-hydrolyzing antibody according to claim 1, wherein the antibody has an amino acid sequence selected from a group consisting of amino acid sequences of SEQ ID NOS: 14 to 24.

6. The nucleic acid-hydrolyzing antibody according to claim 5, wherein the antibody have a base sequence selected from a group consisting of base sequences of SEQ ID NOS: 25 to 35.

7. The nucleic acid-hydrolyzing antibody according to claim 1, wherein the antibody is one selected from a group consisting of an entire IgG, single domain of the heavy chain variable region, single domain of the light chain variable region, single-chain variable fragments (scFv), (scFv)₂, Fab, Fab′, F(ab′)₂, diabody and dsFv, and a combination thereof.

8. A method of preparing the nucleic acid-hydrolyzing antibody of claim 1, comprising:
1) constructing a library of genes on a template of a cell-penetrating nucleic acid-hydrolyzing antibody which lacks substrate specificity;
2) expressing the library gene constructed in step 1) on a cell surface by use of a surface-displaying vector to produce a library of proteins; and
3) selecting from the library of proteins expressed in step 2) a variant which binds specifically to a nucleic acid target of a particular base sequence.

9. The method according to claim 8, wherein the cell-penetrating, nucleic acid-hydrolyzing antibody which lacks substrate specificity is one selected from a group consisting of an entire IgG, single domain of the heavy chain variable region, single domain of the light chain variable region, single-chain variable fragments (scFv), (scFv)₂, Fab, Fab′, F(ab′)₂, diabody and dsFv, and a combination thereof.

10. The method according to claim 8, wherein the cell-penetrating, nucleic acid-hydrolyzing antibody which lacks substrate specificity is 3D8 VL 4M or its variant.

11. The method according to claim 10, wherein the 3D8 VL 4M is mutated in such a manner that a DNA/RNA binding site of 3D8 VL, composed of c- (residues 41-45), c′- (50-54) and f-β-strands (residues 90-94), is randomized with NNB codons (N=A/T/C/G, B=C/G/T).

12. The method according to claim 8, wherein the surface-displaying vector of step 2) is selected from a group consisting of phage display, bacterial display, ribosome display, RNA display and yeast cell display vectors and a combination thereof.

13. The method according to claim 8, wherein the nucleic acid target of step 3) is an endogenous nucleic acid or an exogenous nucleic acid.

14. The method according to claim 13, wherein the endogenous nucleic acid is a nucleic acid coding for a protein overexpressed specifically in cancer cells.

15. The method according to claim 13, wherein the exogenous nucleic acid is a viral genomic nucleic acid or a nucleic acid coding for a viral protein.

16. A composition for prevention or treatment of cancer, comprising the nucleic acid-hydrolyzing antibody of claim 1 as an active ingredient.

17. A composition for prevention or treatment of viral proliferation, comprising the nucleic acid-hydrolyzing antibody of claim 1 as an active ingredient.


SEQUENCE LISTING (41 sequences)

SEQ ID NO: 1 - protein, 113 aa, Artificial Sequence
  amino acid sequence of 3D8 VL WT
DLVMSQSPSSLAVSAGEKVTMSCKSSQSLFNSRTRKNYLAWYQQKPGQSPKLLIYWASTRESGVPDRFTGSGSGTDFTLTISSVQAEDLAVYYCKQSYYHMYTFGSGTKLEIK

SEQ ID NO: 2 - protein, 113 aa, Artificial Sequence
  amino acid sequence of 3D8 VL 4M
DLVMSQSPSSLAVSAGEKVTMSCKSSQSLFNSRTRKNYLAWYQQKPGRSPKLLIHRASTRESGVPDRFTGSGSGTDFTLTISSVQAEDLAVYYCKQSYYAMYTFGSGTKLEIK

SEQ ID NO: 3 - DNA, 339 bp, Artificial Sequence
  nucleotide sequence of 3D8 VL WT
gatcttgtgatgtcacagtctccatcctccctggctgtgtcagcaggagagaaggtcactatgagctgcaaatccagtcagagtctgttcaacagtagaacccgaaagaactacttggcttggtaccagcagaaaccagggcagtctcctaaactgctgatctactgggcatccactagggaatctggggtccctgatcgcttcacaggcagtggatctgggacagatttcactctcaccatcagcagtgtgcaggctgaagacctggcagtttattactgcaagcaatcttattatcacatgtatacgttcggatcggggaccaagctggaaataaaa

SEQ ID NO: 4 - DNA, 339 bp, Artificial Sequence
  nucleotide sequence of 3D8 VL 4M
gatcttgtgatgtcacagtctccatcctccctggctgtgtcagcaggagagaaggtcactatgagctgcaaatccagtcagagtctgttcaacagtagaacccgaaagaactacttggcttggtaccagcagaaaccagggcggtctcctaaactgctgatccaccgggcatccactagggaatctggggtccctgatcgcttcacaggcagtggatctgggacagatttcactctcaccatcagcagtgtgcaggctgaagacctggcagtttattactgcaagcaatcttattatgccatgtatacgttcggatcggggaccaagctggaaataaaa

SEQ ID NO: 5 - DNA, 24 bp, Artificial Sequence
  nucleotide sequence of primer 1F
caggctagtggtggtggtggttct

SEQ ID NO: 6 - DNA, 60 bp, Artificial Sequence
  nucleotide sequence of primer 2R
cagcagtttaggagaccgccctggvnnvnnvnnvnnvnnagccaagtagttctttcgggt

SEQ ID NO: 7 - DNA, 51 bp, Artificial Sequence
  nucleotide sequence of primer 3R
agattccctagtggatgcccggtgvnnvnnvnnvnnvnnagaccgccctgg

SEQ ID NO: 8 - DNA, 24 bp, Artificial Sequence
  nucleotide sequence of primer 4F
caccgggcatccactagggaatct

SEQ ID NO: 9 - DNA, 24 bp, Artificial Sequence
  nucleotide sequence of primer 5R
caggtcttcagcctgcacactgct

SEQ ID NO: 10 - DNA, 60 bp, Artificial Sequence
  nucleotide sequence of primer 6F
agcagtgtgcaggctgaagacctgnnbnnbnnbnnbnnbaagcaatcttattatgccatg

SEQ ID NO: 11 - DNA, 30 bp, Artificial Sequence
  nucleotide sequence of primer 7R
gatctcgcgctattacaagtcctcttcaga

SEQ ID NO: 12 - DNA, 18 bp, Artificial Sequence
  nucleotide sequence of 5′-biotinylated substrate(G(18))
gggggggggggggggggg

SEQ ID NO: 13 - DNA, 18 bp, Artificial Sequence
  nucleotide sequence of 5′-biotinylated substrate(Her2(18))
aattccagtggccatcaa

SEQ ID NO: 14 - protein, 113 aa, Artificial Sequence
  amino acid sequence of 3D8 VL mutant(4MG1)
DLVMSQSPSSLAVSAGEKVTMSCKSSQSLFNSRTRKNYLAWNQRKPGRSRKSLIHRASTREPGVPDRFTGSGSGTDFTLTISSVEPEELAGYYCKQCYYAMYTFGSGTKLEIK

SEQ ID NO: 15 - protein, 113 aa, Artificial Sequence
  amino acid sequence of 3D8 VL mutant(4MG2)
DLVMSQSPSSLAVSAGEKVTMSCKSSQSLFNSRTRKNYLAWQQRKPGRSRKRLIHRASTRESGVPDRFTGSGSGTDFTLTISSVQAEEVGRGGDKQSYYAMYTFGSGTKLEIK

SEQ ID NO: 16 - protein, 113 aa, Artificial Sequence
  amino acid sequence of 3D8 VL mutant(4MG3)
DLVMSQSPSSLAVSAGEKVTMSCKSSQSLFNSRARKNYLAWRQKKPGRSRKQLIHRASTRESGVPDRFTGSGSGTDFTLTISSVQAEELREENRKESYYAMYTFGSGTKLEIK

SEQ ID NO: 17 - protein, 113 aa, Artificial Sequence
  amino acid sequence of 3D8 VL mutant(4MG4)
DLVMSQSPSSLAVSAGEKVTMSCKSSQSLFNSRTRKNYLANNRRRPGRSRNKHEHRASTRESGVPDRFTGSGSGTDFTLTISSVQGEELPEDPHKQSYYAMYTFGSGTKLEIK

SEQ ID NO: 18 - protein, 113 aa, Artificial Sequence
  amino acid sequence of 3D8 VL mutant(4MG5)
DLVMSQSPSSLAVSAGEKVTMSCKSSQSLFNSRTRKNYLAKNQGQPGRSRKNNRHRASTRESGVPDRFTGSGSGTDFTLTISSVQAEDLGRYNSNQSYYAMYTFGSGTKLEIK

SEQ ID NO: 19 - protein, 113 aa, Artificial Sequence
  amino acid sequence of 3D8 VL mutant(4MG6)
DLVMSQSPSSLAVSAGEKVTMSCKSSQSLFNSRTRKNYLASRKRGPGRSGKNHRHRASTRESGVPDRFTGSGSGTDFTLTISSVEGEDLGEYWCKESYYAMYTFGSGTKLEIK

SEQ ID NO: 20 - protein, 113 aa, Artificial Sequence
  amino acid sequence of 3D8 VL mutant(4MH1)
DLVMSQSPSSLAVSAGEKVTMSCKSSQSLFNSRTRKNYLASKEKHPGRSNGSRQHRASTRESGVPDRFTGSGSGTDFTLTISSVQAEELAYYNCKQSYYAMYTFGSGTKLEIK

SEQ ID NO: 21 - protein, 113 aa, Artificial Sequence
  amino acid sequence of 3D8 VL mutant(4MH2)
DLVMSQSPSSLAVSAGEKVTMSCKSSQSLFNSRTRKNYLAWNQCKPGRSEKNLIHRASTRESGVPDRFTGSGSGTDFTLTISSVQAEDLDIQQAKQCYYAMYTFGSGTKLEIK

SEQ ID NO: 22 - protein, 113 aa, Artificial Sequence
  amino acid sequence of 3D8 VL mutant(4MH3)
DLVMSQSPSSLAVSAGEKVTMSCKSSQSLFNSRTRKNYLASERKRPGRSENNRRHRASTRESGVPDRFTGSGSGTDFTLTISSVQAQDLGDQQGKECYYAMYTFGSGTKLEIK

SEQ ID NO: 23 - protein, 113 aa, Artificial Sequence
  amino acid sequence of 3D8 VL mutant(4MH4)
DLVMSQSPSSLAVSAGEKVTMSCKSSQSLFNSRTRKNYLAWYQHKPGRSGKSLIHRASTRESGVPDRFTGSGSGTDFTLTISSVQAEDLGNYGCKECYYAMYTFGSGTKLEIK

SEQ ID NO: 24 - protein, 113 aa, Artificial Sequence
  amino acid sequence of 3D8 VL mutant(4MH5)
DLVMSQSPSSLAVSAGEKVTMSCKSSQSLFNSRTRKNYLAWYQQKPGRSSKGLIHRASTRESGVPDRFTGSGSGTDFTLTISSVQAEELRGKRGKQCYYAMYTFGSGTKLEIK

SEQ ID NO: 25 - DNA, 339 bp, Artificial Sequence
  nucleotide sequence of 3D8 VL mutant(4MG1)
gatcttgtgatgtcacagtctccatcctccctggctgtgtcagcaggagagaaggtcactatgagctgcaaatccagtcagagtctgttcaacagtagaacccgaaagaactacttggcttggaaccagcgcaaaccagggcggtctcgcaaaagcctgatccaccgggcatccaccagggaacctggggtccctgatcgcttcacaggcagtggatctgggacagatttcactctcaccatcagcagtgtggagcctgaagagctggcagggtattactgcaagcaatgttattatgccatgtatacgttcggatcggggaccaagctggaaataaaa

SEQ ID NO: 26 - DNA, 339 bp, Artificial Sequence
  nucleotide sequence of 3D8 VL mutant(4MG2)
gatcttgtgatgtcacagtctccatcctccctggctgtgtcagcaggagagaaggtcactatgagctgcaaatccagtcagagtctgttcaacagtagaacccgaaagaactacttggcttggcagcagcgtaaaccagggcggtctcgcaaacgcctgatccaccgggcatccactagggaatctggggtccctgatcgcttcacaggcagtggatctgggacagatttcactctcaccatcagcagtgtgcaggctgaagaggtgggtcggggtggggacaagcaatcttattatgccatgtatacgttcggatcggggaccaagctggaaataaaa

SEQ ID NO: 27 - DNA, 339 bp, Artificial Sequence
  nucleotide sequence of 3D8 VL mutant(4MG3)
gatcttgtgatgtcacagtctccatcctccctggctgtgtcagcaggagagaaggtcactatgagctgcaaatccagtcagagtctgttcaacagtagagcccgaaagaactacttggcttggaggcagaagaaaccagggcggtctcgcaaacagctgatccaccgggcatccactagggaatctggggtccctgatcgcttcacaggcagtggatctgggacagatttcactctcaccatcagcagtgtgcaggctgaagagctgagggaagaaaaccggaaggaatcttattatgccatgtatacgttcggatcggggaccaagctggaaataaaa

SEQ ID NO: 28 - DNA, 339 bp, Artificial Sequence
  nucleotide sequence of 3D8 VL mutant(4MG4)
gatcttgtgatgtcacagtctccatcctccctggctgtgtcagcaggagagaaggtcactatgagctgcaaatccagtcagagtctgttcaacagtagaacccgaaagaactacttggctaataacaggcgtaggccagggcggtctcggaataaacatgaacaccgggcatccactagggaatctggggtccctgatcgcttcacaggcagtggatctgggacagatttcactctcaccatcagcagtgtgcagggtgaagagctgccggaggatcctcacaagcaatcttattatgccatgtatacgttcggatcggggaccaagctggaaataaaa

SEQ ID NO: 29 - DNA, 339 bp, Artificial Sequence
  nucleotide sequence of 3D8 VL mutant(4MG5)
gatcttgtgatgtcacagtctccatcctccctggctgtgtcagcaggagagaaggtcactatgagctgcaaatccagtcagagtctgttcaacagtagaacccgaaagaactacttggctaaaaatcaaggacaaccagggcggtctagaaaaaacaacaggcaccgggcatccactagggaatctggggtccctgatcgcttcacaggcagtggatctgggacagatttcactctcaccatcagcagtgtgcaggctgaagacctgggacgttataattccaaccaatcttattatgccatgtatacgttcggatcggggaccaagctggaaataaaa

SEQ ID NO: 30 - DNA, 339 bp, Artificial Sequence
  nucleotide sequence of 3D8 VL mutant(4MG6)
gatcttgtgatgtcacagtctccatcctccctggctgtgtcagcaggagagaaggtcactatgagctgcaaatccagtcagagtctgttcaacagtagaacccgaaagaactacttggctagtagaaagcgaggaccagggcggtctggtaagaaccacagacaccgggcatccactagggaatctggggtccctgatcgcttcacaggcagtggatctgggacagatttcactctcaccatcagcagtgtggagggtgaagacctgggagagtattggtgcaaggaatcttattatgccatgtatacgttcggatcggggaccaagctggaaataaaa

SEQ ID NO: 31 - DNA, 339 bp, Artificial Sequence
  nucleotide sequence of 3D8 VL mutant(4MH1)
gatcttgtgatgtcacagtctccatcctccctggctgtgtcagcaggagagaaggtcactatgagctgcaaatccagtcagagtctgttcaacagtagaacccgaaagaactacttggctagtaaggaaaaacacccagggcggtctaacggcagccgacagcaccgggcatccactagggaatctggggtccctgatcgcttcacaggcagtggatctgggacagatttcactctcaccatcagcagtgtgcaggctgaagagctggcatattataactgcaagcaatcttattatgccatgtatacgttcggatcggggaccaagctggaaataaaa

SEQ ID NO: 32 - DNA, 339 bp, Artificial Sequence
  nucleotide sequence of 3D8 VL mutant(4MH2)
gatcttgtgatgtcacagtctccatcctccctggctgtgtcagcaggagagaaggtcactatgagctgcaaatccagtcagagtctgttcaacagtagaacccgaaagaactacttggcttggaaccagtgcaaaccagggcggtctgagaaaaatctgatccaccgggcatccactagggaatctggggtccctgatcgcttcacaggcagtggatctgggacagatttcactctcaccatcagcagtgtgcaggctgaagacctggatattcagcaagcgaagcaatgttattatgccatgtatacgttcggatcggggaccaagctggaaataaaa

SEQ ID NO: 33 - DNA, 339 bp, Artificial Sequence
  nucleotide sequence of 3D8 VL mutant(4MH3)
gatcttgtgatgtcacagtctccatcctccctggctgtgtcagcaggagagaaggtcactatgagctgcaaatccagtcagagtctgttcaacagtagaacccgaaagaactacttggctagtgagcgaaagcgaccagggcggtctgagaataacaggcggcaccgggcatccactagggaatctggggtccctgatcgcttcacaggcagtggatctgggacagatttcactctcaccatcagcagtgtgcaggctcaagacctgggtgatcagcaagggaaggaatgttattatgccatgtatacgttcggatcggggaccaagctggaaataaaa

SEQ ID NO: 34 - DNA, 339 bp, Artificial Sequence
  nucleotide sequence of 3D8 VL mutant(4MH4)
gatcttgtgatgtcacagtctccatcctccctggctgtgtcagcaggagagaaggtcactatgagctgcaaatccagtcagagtctgttcaacagtagaacccgaaagaactacttggcttggtaccagcataaaccagggcggtctggcaaaagtctgatccaccgggcatccactagggaatctggggtccctgatcgcttcacaggcagtggatctgggacagatttcactctcaccatcagcagtgtgcaggctgaagacctgggaaactatggttgcaaggaatgttattatgccatgtatacgttcggatcggggaccaagctggaaataaaa

SEQ ID NO: 35 - DNA, 339 bp, Artificial Sequence
  nucleotide sequence of 3D8 VL mutant(4MH5)
gatcttgtgatgtcacagtctccatcctccctggctgtgtcagcaggagagaaggtcactatgagctgcaaatccagtcagagtctgttcaacagtagaacccgaaagaactacttggcttggtaccagcagaaaccagggcggtctagcaaagggctgatccaccgggcatccactagggaatctggggtccctgatcgcttcacaggcagtggatctgggacagatttcactctcaccatcagcagtgtgcaggctgaagagctgagggggaagcggggcaagcaatgttattatgccatgtatacgttcggatcggggaccaagctggaaataaaa

SEQ ID NO: 36 - DNA, 18 bp, Artificial Sequence
  nucleotide sequence of FRET substrate(A18) which was labeled with 6-FAM at 5′-terminus and BHQ-1 at 3′-terminus
aaaaaaaaaaaaaaaaaa

SEQ ID NO: 37 - DNA, 18 bp, Artificial Sequence
  nucleotide sequence of FRET substrate(T18) which was labeled with 6-FAM at 5′-terminus and BHQ-1 at 3′-terminus
tttttttttttttttttt

SEQ ID NO: 38 - DNA, 18 bp, Artificial Sequence
  nucleotide sequence of FRET substrate(C18) which was labeled with 6-FAM at 5′-terminus and BHQ-1 at 3′-terminus
cccccccccccccccccc

SEQ ID NO: 39 - DNA, 18 bp, Artificial Sequence
  nucleotide sequence of FRET substrate((G4T)3G3) which was labeled with 6-FAM at 5′-terminus and BHQ-1 at 3′-terminus
ggggtggggtggggtggg

SEQ ID NO: 40 - DNA, 18 bp, Artificial Sequence
  nucleotide sequence of FRET substrate(Her2(18)) which was labeled with 6-FAM at 5′-terminus and BHQ-1 at 3′-terminus
aattccagtggccatcaa

SEQ ID NO: 41 - DNA, 18 bp, Artificial Sequence
  nucleotide sequence of FRET substrate(N18) which was labeled with 6-FAM at 5′-terminus and BHQ-1 at 3′-terminus
actgactgactgactgac